(19) United States
(12) Patent Application Publication
(10) Pub. No.: US 2024/0408257 A1
(43) Pub. Date: Dec. 12, 2024

(54) VOLATILE MATERIAL DISPENSER

(71) Applicant: S. C. JOHNSON & SON, INC., Racine, WI (US)

(72) Inventors: Paul E. FURNER, Racine, WI (US); Seth FRANKEL, Arlington, MA (US)

(21) Appl. No.: 18/207,017

(22) Filed: Jun. 7, 2023

(51) Int. Cl. A61L 9/03

(52) U.S. Cl. CPC A61L 9/03; A61L 2209/11; A61L 2209/12; A61L 2209/133; A61L 2209/15

(57) ABSTRACT

A dispensing system includes a dispenser having a front portion that is configured to illuminate one or more illumination elements and a rear portion from which a plug deck extends, the rear portion defining a receiving port that is configured to receive a refill, a heater assembly that is disposed within the rear portion and configured to heat a volatile material disposed in the refill, and a printed circuit board having a controller and being electrically coupled with the heater assembly. The controller is configured to control a temperature of the heater assembly, receive instruction via wireless communication from an electronic device, and communicate an amount of time that the refill has been heated by the heater assembly to the electronic device. The one or more illumination elements are configured to communicate a status of the dispenser to a user based on an operational status of the dispenser.

### BACKGROUND OF THE DISCLOSURE

### 1. Field of the Disclosure

The present disclosure relates generally to methods and systems for dispensing volatile materials, and more particularly, to systems and methods for controlling a volatile material dispenser using an electronic device to improve a user's experience.

### 2. Description of the Background of the Disclosure

Various volatile material dispensers are known in the prior art and generally include a housing with a refill inserted therein. The refill generally includes a container for holding a volatile material, such as a fragrance or pest repellant, therein. In some dispensers, the volatile material is passively emitted therefrom. In other dispensers, a diffusion element is utilized to facilitate the dispensing of the volatile material. Examples of diffusion elements include heaters such as positive temperature coefficient (PTC) heaters, piezoelectric elements, fans, aerosol actuators, and the like. Regardless of the manner in which the volatile material is emitted, once the volatile material has been expended from the refill, the refill is removed by a user and replaced with a new refill.

One type of volatile material dispenser, which is sometimes referred to as a plug-in scented oil dispenser, includes a housing and a heater disposed within the housing. A refill for use with a plug-in scented oil dispenser generally includes a container with a volatile material therein and a wick in contact with the volatile material and extending out of the refill. Upon insertion of the refill into the dispenser, at least a portion of the wick is disposed adjacent the heater such that volatile material that moves through the wick is volatilized by the heater. The volatile material dispenser typically includes a plug assembly having electrical prongs extending outwardly from the housing. The electrical prongs are inserted into a standard electrical outlet and thereafter supply electrical energy to the volatile material dispenser.

Many volatile material dispensers include visual cues, such as individual lights, that provide visual feedback to a user related to aspects of the device such as a current setting or an intensity selection. However, such volatile material dispensers are generally only manually configurable, which can create obstacles for efficient or desirable use thereof. Further, refills for use with such volatile material dispensers are generally visible to a user from multiple viewing angles, which may aid in allowing a user to determine a liquid content of the refill but may otherwise be an undesirable feature in certain implementations of such volatile material devices. As a result, improvements to volatile material dispensers that address one or more of the aforementioned deficiencies are needed.

### SUMMARY OF THE DISCLOSURE

In some embodiments, a dispensing system includes a dispenser having a front portion that is configured to illuminate one or more illumination elements and a rear portion from which a plug deck extends, the rear portion defining a receiving port that is configured to receive a refill. The dispensing system further includes a heater assembly that is disposed within the rear portion and configured to heat a volatile material disposed in the refill and a printed circuit board having a controller and being electrically coupled with the heater assembly, the controller configured to control a temperature of the heater assembly, receive instructions via wireless communication from an electronic device, and communicate an amount of time that the refill has been heated by the heater assembly to the electronic device. The one or more illumination elements are configured to communicate a status of the dispenser to a user based on an operational status of the dispenser.

In some embodiments, the illumination elements are light emitting diodes (LEDs). In some embodiments, the front portion includes a light ring disposed along or within the front portion that includes at least one illumination region. In some embodiments, the light ring includes at least three illumination regions configured to illuminate in a pattern to communicate the status of the dispensing device. In some embodiments, the rear portion includes an illumination region configured to illuminate a surface behind the dispensing device. In some embodiments, the controller calculates an amount of the volatile material in the refill based on the amount of time that the refill has been heated by the heater. In some embodiments, the controller is configured to notify the electronic device when a predetermined amount of volatile material remains in the refill. In some embodiments, the volatile material is one of at least a pest repellant or fragrance. In some embodiments, at least one of a geo-location, a time of day, a season, or a weather forecast is used to determine a temperature of the heater.

In some embodiments, a dispenser includes a front portion that is configured to illuminate three or more illumination elements that are radially offset from and centered about a point, a rear portion from which a plug deck extends, the rear portion defining a receiving port that is configured to receive a refill, a heater assembly that is disposed within the rear portion and is configured to heat a volatile material disposed in the refill, and a printed circuit board having a controller and being electrically coupled with the heater assembly, the controller configured to regulate a temperature of the heater assembly. The three or more illumination elements, in combination, are configured to communicate a status of the device to a user based on a status of the dispenser at least by varying an illumination status of the illumination elements.

In some embodiments, varying the illumination elements includes illuminating the illumination elements in a pattern or altering an illumination color of the illumination elements. In some embodiments, the illumination elements are arranged in an equilateral triangular configuration. In some embodiments, the front portion includes an emblem disposed thereon configured to illuminate to communicate the status to the user based on the status of the dispenser.

In some embodiments, a dispenser includes a front portion that is configured to transmit light emitted by one or more illumination elements, a rear portion from which a plug deck extends, the rear portion defining a receiving port that is configured to receive a refill, a heater assembly that is disposed within the rear portion and configured to heat a volatile material disposed in the refill, a printed circuit board having a controller and being electrically coupled with the heater assembly, the controller configured to regulate a temperature of the heater assembly, and receive instruction via a wireless communication from an electronic device. The refill is not visible when the dispensing device is viewed from a front side thereof when the refill is in thermal communication with the heater assembly.

In some embodiments, the refill is visible when the dispensing device is viewed from a left or right side thereof when the refill is in thermal communication with the heater assembly. In some embodiments, the front portion defines a rounded rectangle periphery. In some embodiments, the rear portion includes a flanged aperture through which volatile material is configured to release once it has been heated. In some embodiments, the housing includes one or more rings of dimples at least partially surrounding the flanged aperture. In some embodiments, the heater assembly is aligned with the flanged aperture. In some embodiments, the controller is configured to regulate the temperature of the heater assembly based on a size of a room where the dispensing device is located.

### BRIEF DESCRIPTION OF THE DRAWINGS

FIG. 1 is front, left, isometric view of a dispensing system having a dispenser and an electronic device in accordance with the present disclosure;

FIG. 2 is a front plan view of the dispenser of the dispensing system of FIG. 1;

FIG. 3 is a left side plan view of the dispenser of FIG. 2;

FIG. 4 is a top plan view of the dispenser of FIG. 2;

FIG. 5 is a rear plan view of the dispenser of FIG. 2;

FIG. 6 is a rear cross-sectional view of the dispenser taken through line 6-6 of FIG. 4;

FIG. 7 is a left side cross-sectional view of the dispenser taken through line 7-7 of FIG. 4;

FIG. 8 is a rear, right perspective view of a housing, PCB, heater assembly, and a refill of the dispenser of FIG. 2;

FIG. 9 is an exploded, isometric view of the dispenser of FIG. 2;

FIG. 10 is a schematic view of various electrical and communication components of the dispensing system of FIG. 1;

FIG. 11 is an example graphical user interface for use on the electronic device of the dispensing system of FIG. 1;

FIG. 12 is an example dispenser graphical user interface that is configured to provide information to a user regarding a status of the dispenser of FIG. 1;

FIG. 13 is an example heating profile for the dispensing system of FIG. 1 configured to a “child” mode;

FIG. 14 is an example heating profile for the dispensing system of FIG. 1 configured to a “standard” mode;

FIG. 15 is an example heating profile for the dispensing system of FIG. 1 configured to a “high” mode;

FIG. 16 is an example method for dispensing a volatile material from a dispenser in accordance with the present disclosure;

FIG. 17 is an example method for connecting a dispenser to an electronic device in accordance with the present disclosure;

FIG. 18 is an example method for dispensing a volatile material from a dispenser based on a weather forecast in accordance with the present disclosure;

FIG. 19 is an example method for calculating an amount of volatile material dispensed by a dispenser in accordance with the present disclosure; and

FIG. 20 is an example method for calculating a heating profile for a dispenser in accordance with the present disclosure.

### DETAILED DESCRIPTION OF THE DRAWINGS

The present disclosure is directed to the control of volatile material dispensers using an electronic device. While the present invention may be embodied in many different forms, several specific embodiments are discussed herein with the understanding that the embodiments of the present disclosure are intended to be considered only exemplifications of the principles of the invention, and are not intended to limit the scope of the invention disclosed herein.

Consumer products are rapidly evolving and gaining the ability to connect wirelessly to electronic devices capable of internet or Bluetooth communication (e.g., phones, computers, Google® Home™, Amazon® Alexa™, etc.). As disclosed herein, allowing users to wirelessly communicate with a dispensing device provides the user with the opportunity to modify dispensation characteristics of the dispensing device with greater speed and convenience. With one or more clicks of a soft button of a software application, dispensation characteristics of the wirelessly-connected dispensing device can be changed from virtually anywhere, providing the user with greater control over dispensation characteristics of their dispenser, such as a release rate or a duration of release of a volatile material.

As discussed in greater detail below, the electronic device may be configured to wirelessly control a rate of energy transfer of a heater assembly to a volatile material. The differences in functionality of the volatile material dispenser and the volatile material may be attributed to different settings available for modification by the user. The setting(s) selected by the user may further be influenced by factors such as a presence of a child, presence of a pet, time of day, season, location of the dispensing device, location of the user, size of the room in which the dispenser is located, and even the weather. The differences in the setting selected by the user may affect the rate, duration, or dispensation pattern or characteristics of the dispensing device. Thus, a variety of different control techniques are used to dictate the energy transfer rate from the heater to the volatile material absorbed by the wick. By regulating the energy applied to a volatile material, the volatile material concentration or intensity characteristics from the volatile material in a first environment can be controlled and adjusted, which can improve a user's experience. The systems and methods disclosed herein allow the volatile material dispensing system to control an energy transfer rate of the heater assembly to the volatile material, and to thus control a volatile material concentration in an environment.

By controlling the energy transfer rate of the heater assembly in a targeted fashion, the volatile material dispenser can adjust or modify the volatile material evaporation rate depending upon user settings, environmental factors, and location characteristics. Thus, the volatile material dispenser may manipulate the volatile material evaporation rate and evaporation process to adjust, for example, a repellent or pesticide to meet the pest reduction needs of the user. The systems and methods herein address an ability of the user to alter the rate of volatile material dispensation using the electronic device, e.g., a phone, tablet, desktop, etc. Throughout the disclosure, the terms “about” and “approximately” refer to a range of values±5% of the numeric value that each term precedes. As noted herein, all ranges disclosed within this application are inclusive of the outer bounds of the range.

The term “volatile material,” as used herein, refers to any substance or a mixture of substances, and may include one or more of a cleaner, an insecticide, an insect repellent, an insect attractant, a disinfectant, a mold or mildew inhibitor, an antimicrobial, a fragrance comprised of one or more aroma chemicals, a disinfectant, an air purifier, an aromatherapy scent, an antiseptic, an odor eliminator, a positive fragrance active material, an air-freshener, a deodorizer, a medicinal component, an inhalant (e.g., for relieving a cough or congestion), or the like, and combinations thereof. The volatile material compositions of the present application may comprise a single chemical or may comprise a sophisticated complex mixture of natural and/or synthetic chemical components, all chosen to provide any desired odor or effect.

Although the heater arrangements herein are described as being utilized with dispensers that utilize liquid electric refills, the heater arrangements may be utilized for any electrical dispenser from which any type of volatile material is dispensed out of any type of refill by way of a heater, e.g., scented oils, insect repellants, pesticides, etc. Optionally, a dispenser employing any of the heater arrangements disclosed herein may further include one or more heaters and/or additional devices for dispensing the volatile material, for example, one or more of a fan, a piezoelectric element, and/or other components disposed in a housing thereof to help facilitate the release of the volatile material.

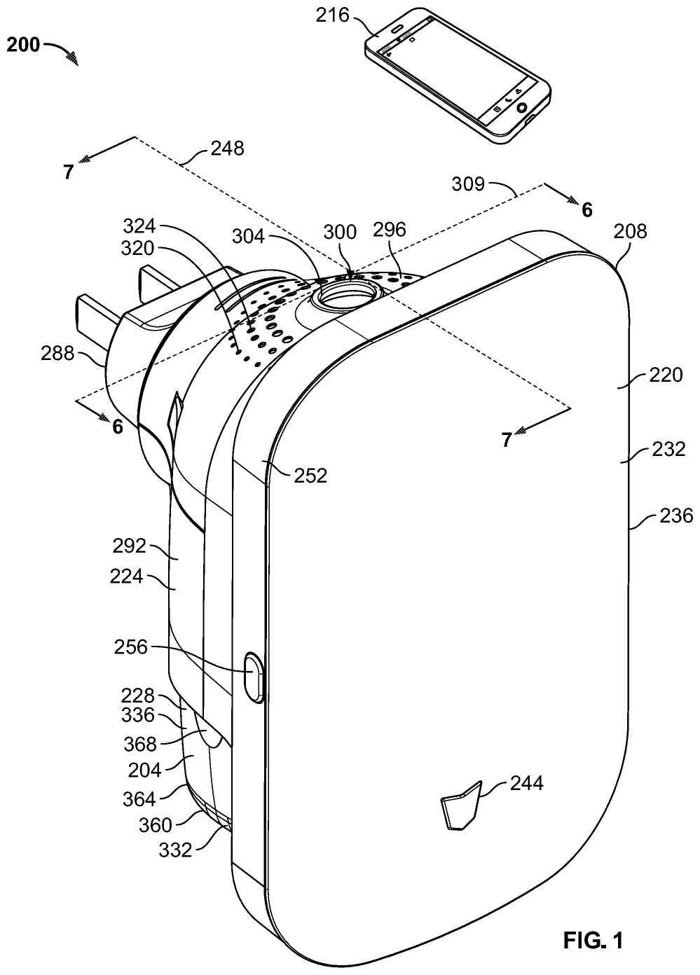

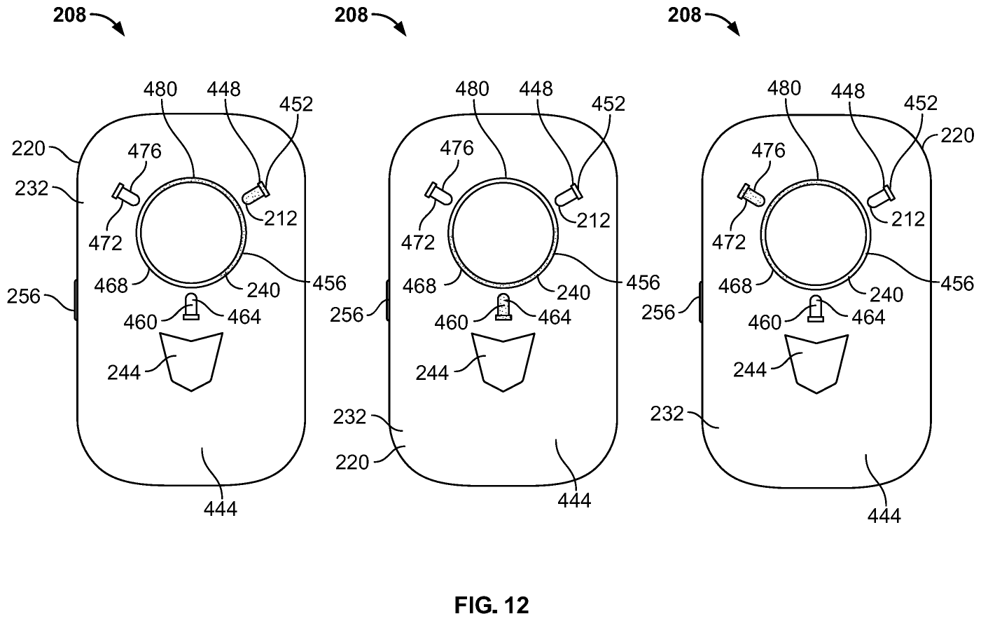

Referring now to the figures, FIGS. 1-8 generally depict a dispensing system 200 and components thereof for use in the emanation of a volatile material 204 into the ambient environment via a dispenser 208. The dispenser 208 is configured to emanate the volatile material 204, which may comprise a fragrance or a pest control agent. The dispenser 208 advantageously provides visual feedback to the user that the device is operating through alternating illumination of one or more illumination elements 212 (representative visual feedback shown in FIG. 12). Referring in particular to FIG. 1, the dispensing system 200 includes the dispenser 208 and an electronic device 216 (e.g., a user device such as a smartphone, smart tablet, computer, smart watch, or other known computing device). The dispenser 208 and the electronic device 216 may be in wireless communication, e.g., via Bluetooth® Low Energy (BLE) wireless communication, as discussed in greater detail hereinafter below. The dispenser 208 is configured to receive commands from the electronic device 216 to alter release characteristics of the volatile material, such as the release rate and schedule of release.

Still referring to FIG. 1, the dispenser 208 comprises a front portion or housing 220, which is disposed at a forward end of the dispenser 208. The dispenser 208 also comprises a rear portion or body 224, which extends rearward from the front portion 220 and is entirely concealed by the front portion 220 when the dispenser 208 is viewed from the front. In some embodiments, the rear portion 224 is coupled directly to the front portion 220. In other embodiments, the rear portion 224 and the front portion 220 are integrally formed. During use of the dispenser 208, a refill 228 is disposed within the rear portion 224 and is configured to be coupled to and decoupled from the rear portion 224. The front portion 220 includes a front wall 232 that curves rearward toward the rear portion 224, although in some embodiments the front wall 232 may be flat. A sidewall 236 extends rearward from the front wall 232 and defines a front wall peripheral shape. In some embodiments, the front wall peripheral shape is a rounded rectangle, a rectangle with rounded corners, or may be any other shape (e.g., an oval, a rectangle, a trapezoid, a hexagon, an octagon, etc.). The front wall 232 may be opaque, transparent, or translucent. In some embodiments, the front wall 232 or a portion thereof is partially transparent and is configured to allow one or more of the illumination elements 212 to shine through the front wall 232 to indicate a status of the dispenser 208 to a user.

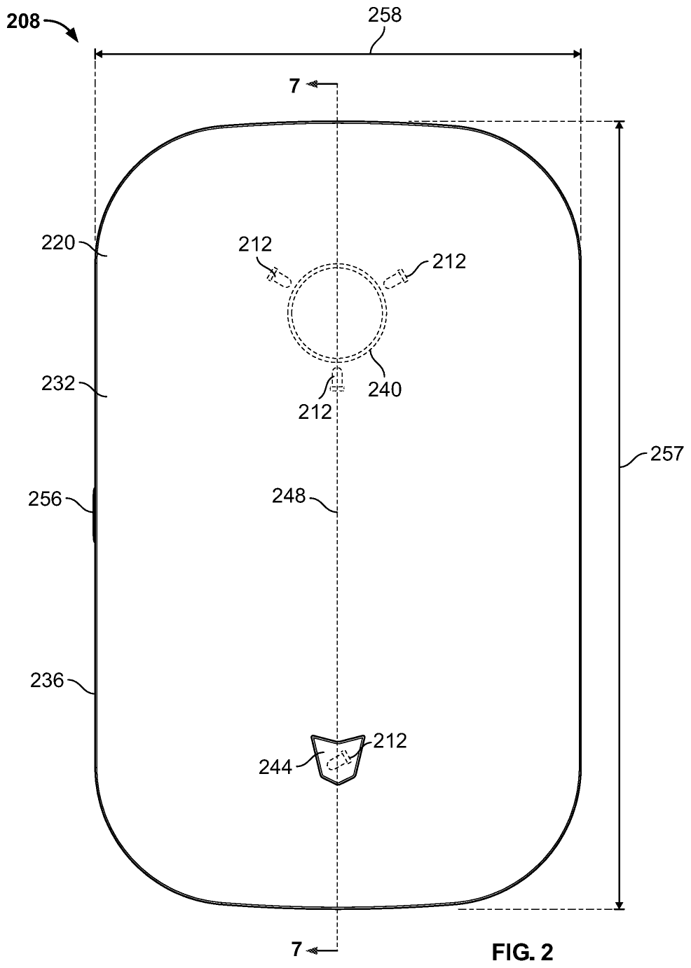

Referring to FIGS. 2 and 12, a light ring 240 (see FIG. 12) comprising one or more of the illumination elements 212 is configured to provide illumination through the front wall 232. In some embodiments, the light ring 240 is defined by a different type of material than the front wall 232 and in the form of a circle that is disposed along or within the front wall 232. In alternative embodiments, the light ring 240 comprises a light pipe that is disposed entirely within the front portion 220, and the light ring 240 is only visible when the one or more illumination elements 212 are illuminated, i.e., the front wall 232 is translucent and allows light therethrough that is transmitted from the light ring 240. In some embodiments, the light ring 240 comprises a light pipe or waveguide that is partially disposed along the front wall 232 and partially disposed within the front portion 220. The light ring 240 is configured to be illuminated based on a status of the dispenser 208, as discussed below, and includes any number of the illumination elements 212, which are configured to alternate between “on” and “off” states to indicate the status of the dispenser 208. The illumination elements 212 of the light ring 240 may further be configured to change color to indicate the status of the dispenser 208. In some embodiments, the user selects a brightness and color of the illumination elements 212 of the light ring 240.

Referring specifically to FIGS. 1 and 2, the front wall 232 further includes an emblem 244, which may be any shape (e.g., a shield, an oval, a rectangle, a trapezoid, a hexagon, an octagon, etc.) or combination of shapes. The emblem 244 may be located along a center plane 248 that bifurcates the front wall 232 in half vertically. In some embodiments, the emblem 244 extends forward from the front wall 232. In some embodiments, the emblem 244 is flush with the front wall 232. The emblem 244 is illuminated via the one or more illumination elements 212, and is configured to indicate the status of the dispenser 208. The emblem 244 includes any number of the illumination elements 212, which are configured to alternate between “on” and “off” states to indicate the status of the dispenser 208 or to otherwise provide an aesthetic benefit. The illumination elements 212 of the emblem 244 may further be configured to change color to indicate the status of the dispenser 208. In some embodiments, the user may select a brightness and color of the illumination elements 212 of the emblem 244.

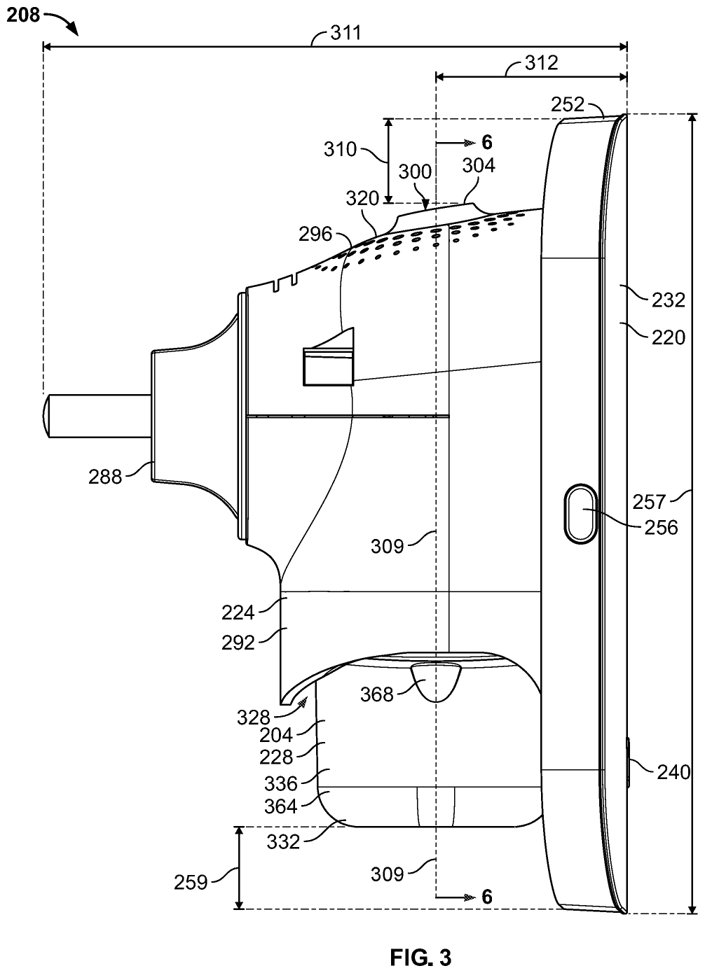

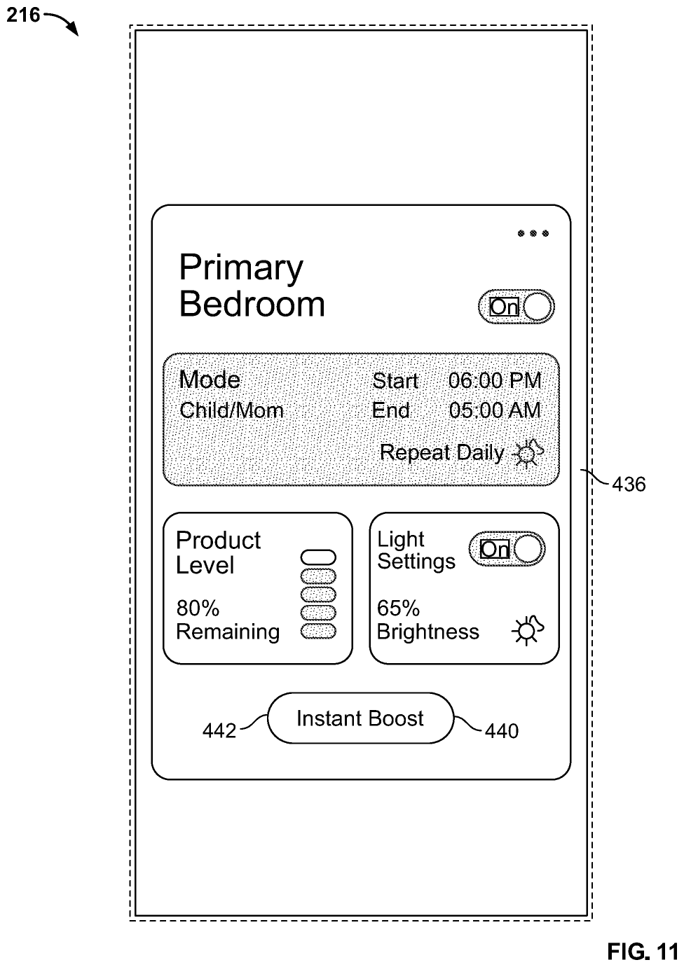

Referring to FIGS. 1 and 3, the front portion 220 includes a side wall 252. The side wall 252 extends rearward of the front wall 232 and includes a button 256 disposed therealong. In some embodiments, the button 256 is configured to initiate a pairing sequence to pair the dispenser 208 with the electronic device 216 once the button 256 is depressed. The dispenser 208 then pairs with the electronic device 216 once the user selects one or more soft buttons on the electronic device 216 (see FIG. 11). In some embodiments, the button 256 is configured to alter the release characteristics of the volatile material 204. In some embodiments, once pressed the button 256 is configured to begin a connection sequence to connect the dispenser 208 to a wireless network. Still further, in some embodiments, once pressed the button 256 is configured to begin a diagnostic sequence for the dispenser 208.

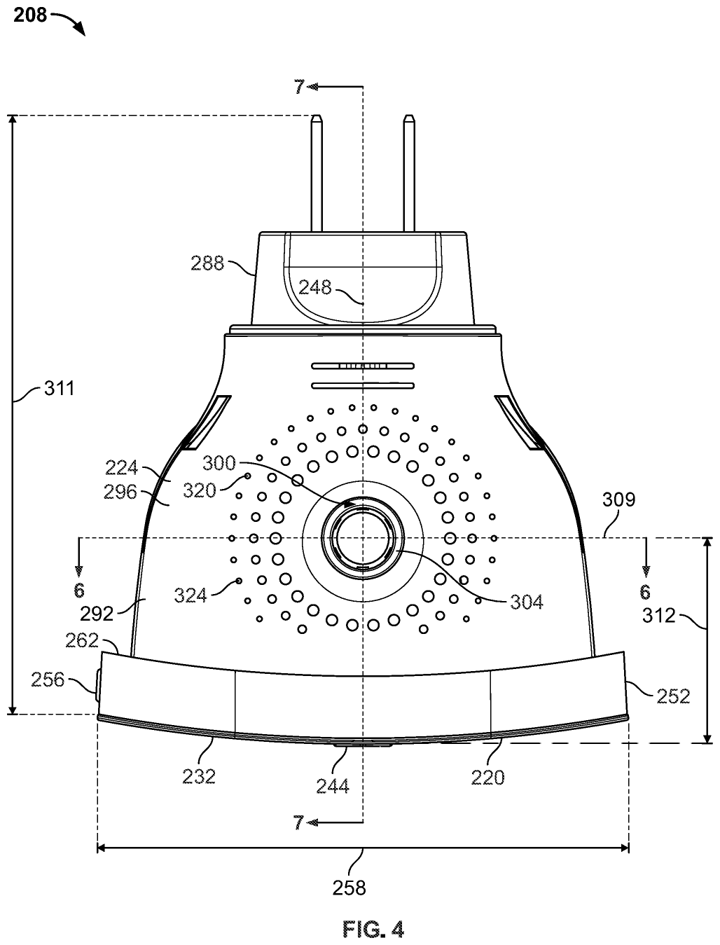

Referring to FIGS. 2-5, the dispenser 208 defines a height 257, shown in FIG. 3, and a width 258, shown in FIG. 4. The height 257 is shown as a line segment that extends from an uppermost surface of the dispenser 208 to a lowermost surface of the dispenser 208, parallel to the center plane 248. The width 258 is also shown as a line segment that extends from a left most surface of the dispenser 208 to a right-most surface of the dispenser 208, measured in a direction that is perpendicular with respect to the center plane 248. The height 257 and the width 258 are discussed below with respect to other aspects and dimensions of the dispenser 208.

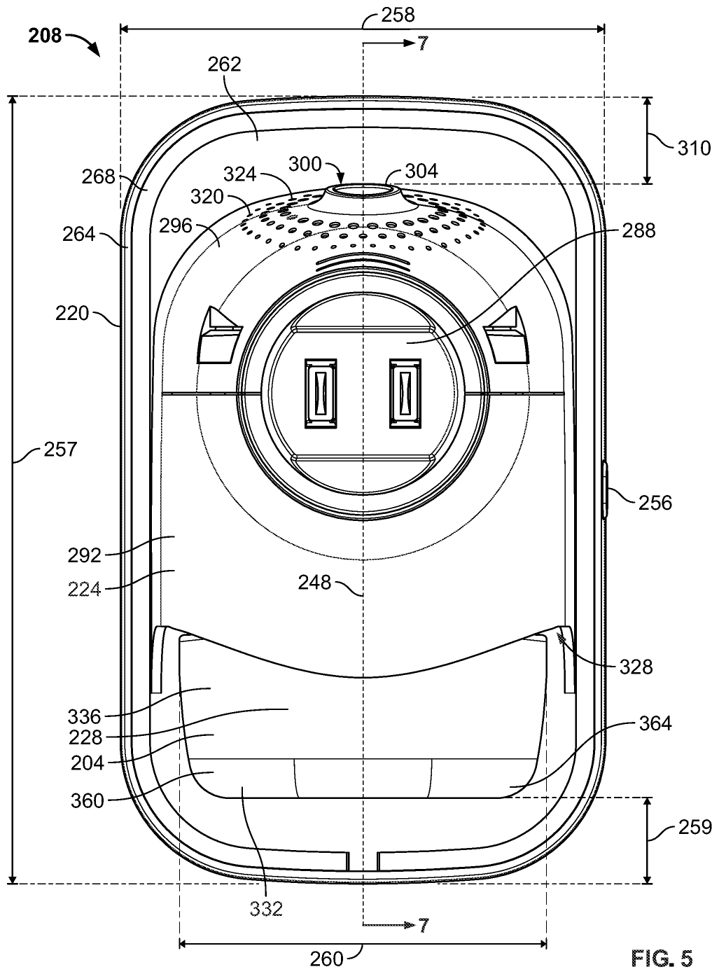

Referring in particular to FIGS. 2 and 3, the rear portion 224 extends rearward of a rear wall 262 of the front portion 220. From a front viewing position, the rear portion 224 and the refill 228 are concealed by the front portion 220. In other words, when the dispenser 208 is in use, the refill 228 and the rear portion 224 are not visible to the user from a front viewing position that is directly in front of the dispenser and at least one meter away. When the dispenser 208 is in use, a bottom of the refill 228 is disposed above a bottom wall of the front portion 220. A clearance height 259 is shown as a line segment, the clearance height 259 being measured from the lowermost surface of the dispenser 208 to a lowermost surface of the refill 228, parallel to the center plane 248. The clearance height 259 is illustrated as being about 15% of the total height 257. In some embodiments, the clearance height 259 may be about 5%, about 10%, about 20%, about 30%, about 40%, about 50%, about 60%, about 70%, about 80%, or about 90% of the total height 257. Referring specifically to FIG. 5, a refill width 260 is shown as a line segment, the refill width 260 being measured from a left-most refill surface to a right-most refill surface, perpendicular to the center plane 248. The refill width 260 is illustrated being about 75% of the width 258. In some embodiments, the refill width 260 may be about 10%, about 20%, about 30%, about 40%, about 50%, about 60%, about 70%, about 80%, about 90%, or about 95% of the width 258.

Referring now to FIGS. 4 and 5, the front portion 220 includes the rear wall 262. The rear wall 262 extends from the side wall 252 and is substantially parallel with respect to the front wall 232. In some embodiments, a rear edge 264 defines a rear wall peripheral shape, which may be the same shape as the front wall peripheral shape. In some embodiments, the rear wall peripheral shape is a different shape from the front wall peripheral shape, and may define any shape (e.g., an oval, a rectangle, a trapezoid, a hexagon, an octagon, etc.). A peripheral region 268 extends inward from the rear edge 264. One or more of the illumination elements 212 are configured to emit light to illuminate the peripheral region 268 based on a state or configuration of the dispenser 208. The peripheral region 268 includes any number of the illumination elements 212, which are configured to alternate between “on” and “off” states to indicate a status of the dispenser 208 to the user. The illumination elements 212 of the peripheral region 268 may further be configured to change color to indicate the status of the dispenser 208, as discussed in greater detail below. In some embodiments, the peripheral region 268 includes a wave guide or light pipe (not shown) configured to illuminate and alternate between “on” and “off” states to indicate a status of the dispenser 208 to the user. The wave guide of the peripheral region 268 also may be configured to change color to indicate the status of the dispenser 208.

In some embodiments, the peripheral region 268 is configured to act as a night light and may illuminate a surface that is disposed behind the dispenser 208, such as a wall (not shown). In some embodiments, the user may select a brightness and color of the illumination elements 212 that are configured to illuminate the peripheral region 268. In some embodiments, the peripheral region 268 may be illuminated if the dispenser 208 senses the presence of the user. In some embodiments, the dispenser 208 senses the presence of the user via a proximity sensor that may be configured to sense motion or a change of light and cause the peripheral region 268 to illuminate. In some embodiments, the dispenser 208 senses the proximity of the electronic device 216 via the wireless connection between the dispenser 208 and the electronic device 216. For example, the dispenser 208 may sense when the electronic device 216 is within a certain radius, e.g., about three meters, or about five meters, or about seven meters, or about 10 meters, or about 13 meters, or about 15 meters, and illuminates the peripheral region 268.

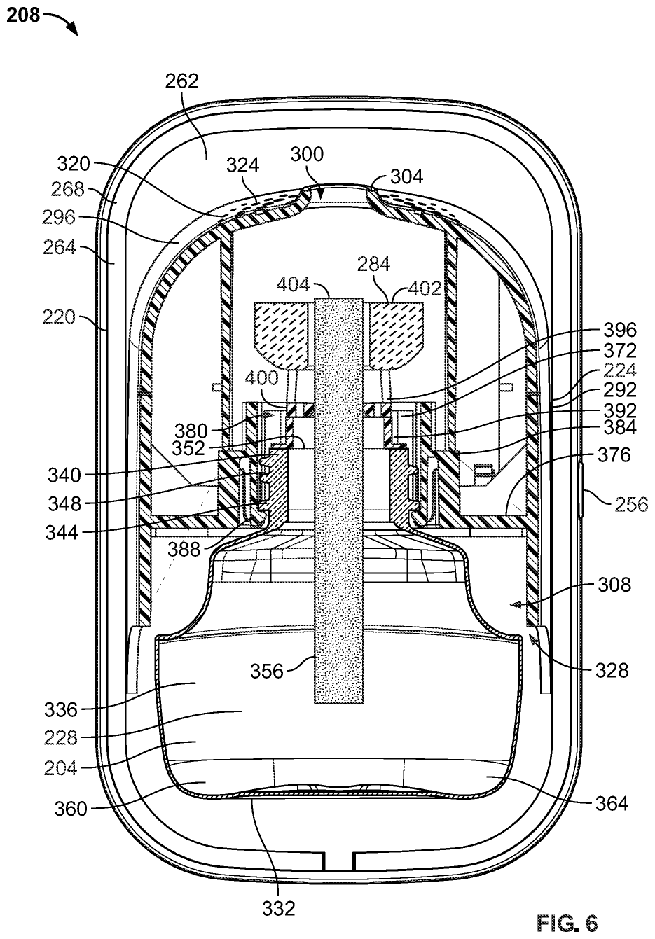

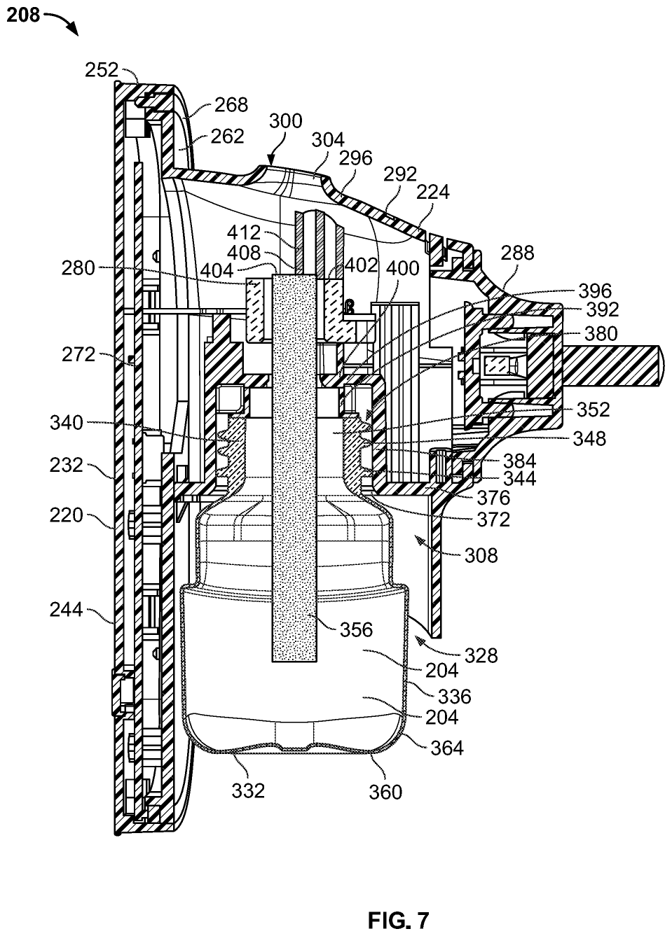

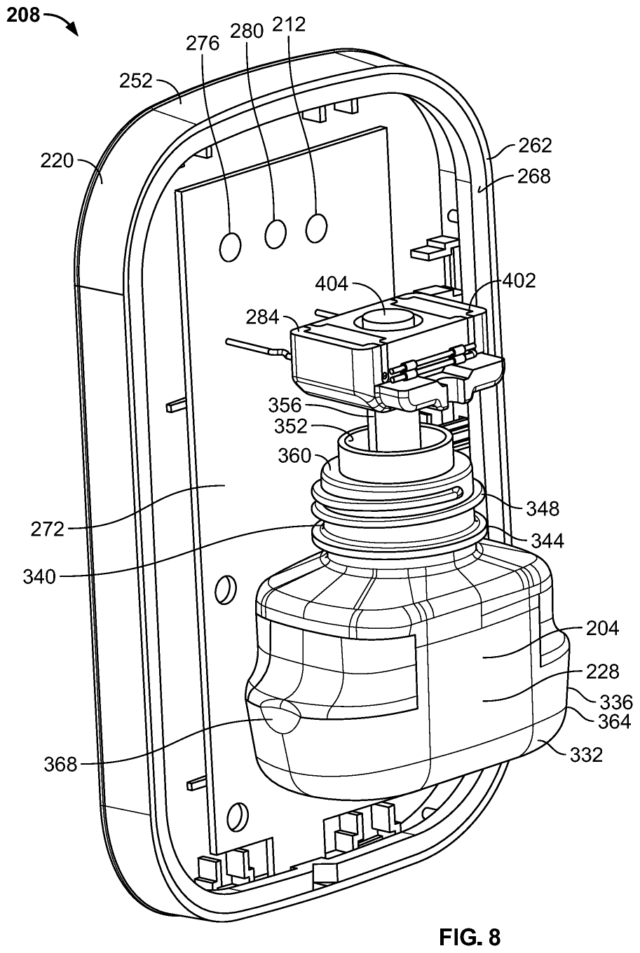

Referring to FIGS. 6 and 7, cross-sectional views of the dispenser 208 are shown, which highlight that the front portion 220 is generally hollow and includes various components of the dispenser 208 disposed therein. In particular, a printed circuit board 272 is disposed within the front portion 220, which is hereinafter referred to as a “PCB” 272. In some embodiments, the PCB 272 includes a controller 276, a wireless communication unit 280, a heater assembly 284, and the one or more illumination elements 212 (see FIG. 8) electrically coupled thereto. Referring specifically to FIG. 7, the heater assembly 284 extends rearward from the PCB 272 toward the rear portion 224. Further, the rear portion 224 includes a plug deck 288 that extends from a rearmost end of the rear portion 224. The plug deck 288 is configured to plug into a wall outlet. In some embodiments, the plug deck 288 is configured to plug into a car or boat outlet. The plug deck 288 is in electrical communication, e.g., using a wired connection or a wireless connection, with the PCB 272 to provide power to electrical components connected to the PCB 272.

Referring to FIGS. 3-7, the rear portion 224 includes a body wall 292, which extends from the rear wall 262. The body wall 292 is generally curved and defines at least two inflection points as it extends toward the plug deck 288. The body wall 292 includes a top portion 296 that defines an aperture 300 through which the volatile material 204 is configured to be dispensed. More specifically, a flange 304 extends upward from the top region 296 and surrounds the aperture 300. The aperture 300 is configured to provide fluid communication between a cavity 308 disposed within the rear portion 224 and the surrounding environment. A longitudinal plane 309 bifurcates the aperture 300 and extends perpendicular with respect to the center plane 248.

Referring specifically to FIG. 3, a flange height 310 is shown, which extends from the uppermost surface of the dispenser 208 to an uppermost flange surface in a direction that is parallel with respect to the center plane 248. The flange height 310 is illustrated being about 15% of the total height 257. In some embodiments, the flange height 310 may be about 5%, about 10%, about 20%, about 30%, about 40%, about 50%, about 60%, about 70%, about 80%, or about 90% of the total height 257. The dispenser 208 also defines a dispenser depth 311, which is shown as a line that extends from the front wall 232 to a rear-most plug deck surface. A flange depth 312 is also shown as a line segment, the flange depth 312 being measured from a frontmost portion of the front wall 232 to a center of the aperture 300, perpendicular to the longitudinal plane 309. The flange depth 312 is illustrated being about 40% of the dispenser depth 311. In some embodiments, the flange depth may be about 20%, about 30%, about 50%, about 60%, about 70%, about 80%, about 90%, or about 95% of the dispenser depth 311.

Referring now to FIG. 4, an outer surface of the top portion 296 defines a plurality of dimples 320 that concentrically surround the aperture 300. In some embodiments, the plurality of dimples 320 are arranged in a circular pattern on the top portion 296. The plurality of dimples 320 may include dimples of different sizes and shapes (e.g., circular, ovular, etc.). In some embodiments one or more of the plurality of dimples 320 may be apertures or holes that extend through the top portion 296. In some embodiments, the plurality of dimples 320 form one or more dimple rings 324 that at least partially surround the aperture 300. In the depicted embodiment, the dimple rings 324 farther from the aperture 300 have a larger radius than the dimple rings 324 that are closer to the aperture, although alternative configurations are contemplated.

Referring to FIGS. 5-7, the rear portion 224 further defines a refill opening 328 at a lower end thereof. The refill opening 328 is disposed at the bottom of the rear portion 224 and is configured to receive the refill 228. The refill opening 328 is fluidly connected to the cavity 308 such that air is capable of flowing through the refill opening 328 into the cavity 308. Before use of the dispenser 208, the refill 228 is inserted through the refill opening 328 into the cavity 308, and the refill is retained by the dispenser 208 until and during operation of the dispenser 208. The refill 228 is removable from the dispenser 208 allowing for the refill 228 to be interchanged. In some embodiments, the dispenser 208 is configured to receive one or more different sizes of the refill 228.

Referring to FIGS. 6 and 8, the refill 228 is configured to hold the volatile material 204. The refill includes a bottom wall 332, a body 336 extending from the bottom wall 332, a neck 340 extending from the body 336, a transfer bead 344 disposed on the neck 340, threading 348 disposed along the neck 340, a refill opening 352 defined by the neck 340, and a wick 356 extending through the refill opening 352. In some embodiments, the bottom wall 332 defines a periphery 360 defining a rounded rectangular shape. The bottom wall 332 includes rounded edges 364 that continuously connect the bottom wall 332 to the body 336. The body 336 extends upward from the bottom wall 332 to the neck 340 of the refill 228. When viewed from either side, the bottom wall 332 and a part of the body 336 are visible when the refill 228 is coupled with the rear portion 224. The body 336 includes finger tabs 368 that allow the user to more effectively grip the refill 228 to decouple the refill 228 from the dispenser 208. In some embodiments, the bottom wall 332 and the body 336 are transparent or translucent to allow a user to see how much volatile material 204 remains in the refill 228. In some embodiments, the bottom wall 332 and the body 336 are opaque.

Referring now to FIGS. 6 and 7, an interior of the rear portion 224 is shown more clearly in cross section. A receiving port 372 is shown being disposed within the cavity 308 and directly above the refill opening 328. The receiving port 372 is configured to receive the refill 228. A bottom of the receiving port 372 is at least partially defined by a platform 376 that extends from the body wall 292 toward the receiving port 372. The platform 376 surrounds the receiving port 372, and the receiving port 372 further defines a receiving region 380. The receiving port 372 includes a receiving port wall 384 which extends upward from the platform 376 to at least partially define the receiving region 380. The receiving region 380 is configured to house at least the neck 340 of the refill 228 before and during use of the dispenser 208.

Referring in particular to FIG. 6, the refill 228 is retained within the receiving port 372 via one or more retention members 388 that extend downward from the receiving port wall 384. The retention members 388 are configured to retain the refill 228 within the receiving port 372 via a snap-fit connection. To couple the refill 228 with the dispenser 208, a user inserts the neck 340 into the receiving port 372 and pushes upward with a force large enough to separate and engage the retention members 388. In some embodiments, the retention members 388 are hooks configured to couple with the transfer bead 344 disposed on the neck 340 of the refill 228. In some embodiments, the retention members 388 extend from left and right sides of the receiving port wall 384 and are configured to deflect outward to accept the transfer bead 344. Once the transfer bead 344 is coupled with the retention members 388, a top surface of the retention member 388 engages a bottom surface of the transfer bead 344 to retain the refill 228 within the receiving port 372. The retention members 388 are configured to be coupled to and decoupled from the transfer bead 344 to allow the refill 228 to be inserted into and thereafter removed from the receiving port 372. In some embodiments, a large enough downward pulling force applied by the user is capable of decoupling the refill 228 from the retention members 388. In some embodiments the downward pulling force applied by the user causes the retention members 388 to deflect outward to decouple the transfer bead 344.

Referring again to FIGS. 6 and 7, a bottle stopper 392 is disposed within the receiving port 372 and is configured to prevent upward translation of the refill 228 beyond a desired distance. The bottle stopper 392 is configured to receive the wick 356 such that the wick 356 extends beyond the bottle stopper 392. Further, the bottle stopper 392 engages the refill opening 352 and is configured to create a mechanical seal with the refill opening 352 to reduce the risk for leakage or excess evaporation of the volatile material 204. In some embodiments, the refill 228 may be retained within the receiving port 372 via a different coupling mechanism. In some embodiments, the refill 228 is screwed into the receiving port 372 via the threading 348. In some embodiments the refill 228 is retained by the receiving port 372 using a press fit (not depicted).

Referring now to FIGS. 6-8, the wick 356 is shown in detail, which is configured to be heated by the heater assembly 284 to volatilize the volatile material 204 into the surrounding environment through the aperture 300. The receiving port 372 is also shown, which includes a wick receiving port 396 disposed in a receiving port ceiling 400. The wick receiving port 396 is configured to receive the wick 356, which extends from the refill 228. As noted above, the heater assembly 284 extends from the PCB 272, toward the rear of the front portion 220. A heating element 402 of the heater assembly 284 is disposed above the wick receiving port 396, which is configured to receive the wick 356. The wick 356 is in fluid communication with the volatile material 204 in the refill 228, and the volatile material 204 travels upward from the refill 228 to a wick tip 404. The heater assembly 284 at least partially surrounds the wick tip 404 and may be in direct contact with the wick tip 404. The volatile material 204 may then be heated by the heater assembly 284, evaporated, and emanated through the aperture 300 in the top portion 296.

Still referring to FIGS. 6-8, the receiving port 372, the wick receiving port 396, and the heater assembly 284 are disposed below the aperture 300 and aligned with a center thereof. In some embodiments, the receiving port 372, the wick receiving port 396, and the heater assembly 284 are disposed along the center plane 248. The positioning of the wick receiving port 396 below the aperture 300 allows for more efficient fluid transfer of the volatile material 204 from the cavity 308 to the surrounding environment. In some embodiments, the wick receiving port 396 and the heater assembly 284 are disposed below the aperture 300 but are not aligned with the aperture 300.

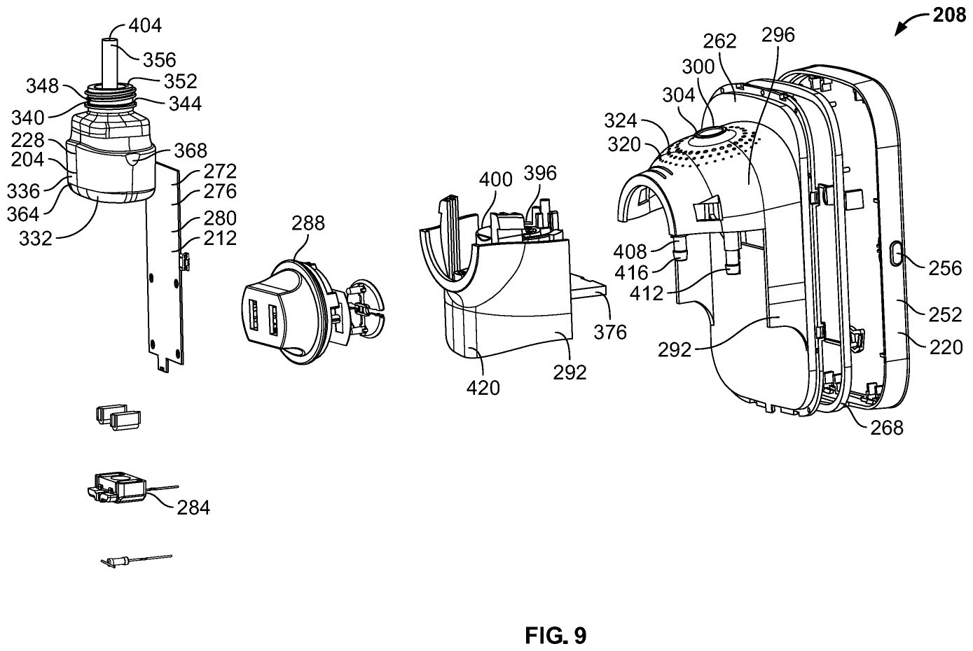

Referring now to FIGS. 7 and 9, one or more columns 408 are shown, which provide support for the rear portion 224. The one or more columns 408 extend from the platform 376 to the top portion 296. The one or more columns 408 aid the rear portion 224 in withstanding compressive forces associated with the insertion of the refill 228. In some embodiments, the one or more columns 408 are situated to a left side and to a right side of the receiving port 372. In some embodiments, a first column 412 is situated on the left side of the receiving port 372, and a second column 416 is situated on a right side of the receiving port 372.

Referring specifically to FIG. 9, an exploded view of an embodiment of the dispenser 208 is shown. In the depicted embodiment, the exploded view highlights which components of the dispenser 208 are integrally formed and which components are separable. In some embodiments, the front wall 232 and side wall 252 of the front portion 220 are integrally formed. In some embodiments, the PCB 272 is coupled to the front wall 232 and/or to the side wall 252. In some embodiments, the side wall 252 is coupled to the peripheral region 268. In some embodiments, the peripheral region 268 is coupled to the rear wall 262. In some embodiments, the rear wall 262 is integrally formed with the rear portion 224. In some embodiments, the rear wall 262 is coupled to the rear portion 224. In some embodiments, the rear portion 224 includes a detachable portion 420 that is coupled to the rear portion 224, which includes the platform 376 and the receiving port 372. The detachable portion 420 is coupled to the plug deck 288. Each component listed above may be coupled via various mechanisms (e.g., snap-fit, press-fit, sliding-fit, spring tabs, hooks, etc.). In some embodiments, any component of the dispenser 208 is configured to be removed and replaced by another component. For example, a plug deck configured to plug into a wall socket, may be replaced by a plug deck configured to plug into a car socket. Though the components mentioned in this paragraph are described as separable or integrally formed, it is contemplated that each component mentioned can be either separable from or integrally formed with any other component.

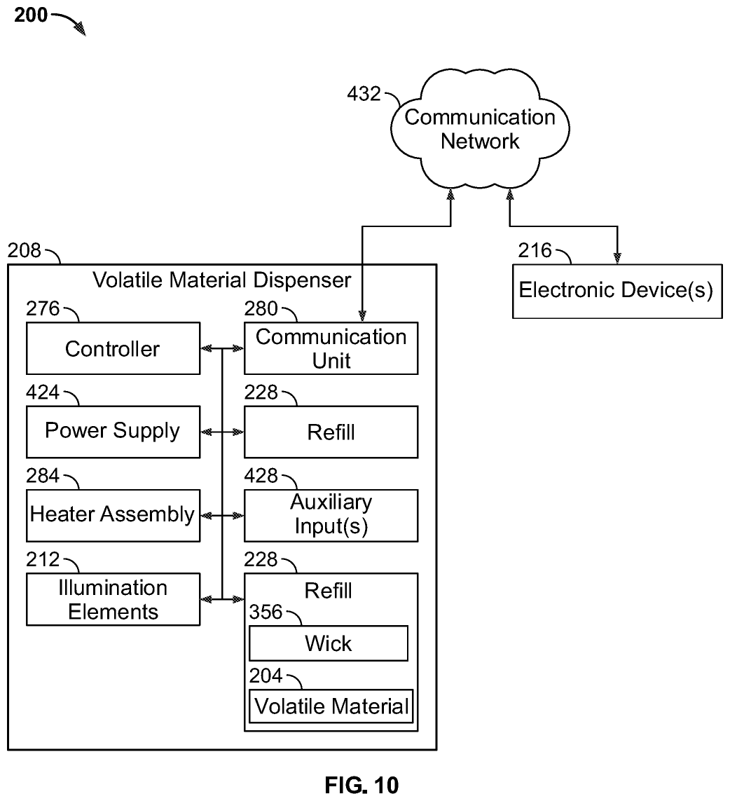

Referring to FIG. 10, a schematic representation of the dispenser 208 of FIG. 1 is shown. The dispenser 208 comprises the controller 276 that is connected to a power supply 424, the heater assembly 284, the plurality of illumination elements 212, the wireless communication unit 280, and the refill 228. The refill 228 includes the volatile material 204. The refill 228 also comprises the fluid delivery system, shown as the wick 356, that is in fluid communication with the volatile material 204 and thermal communication with the heater assembly 284. The heater assembly 284 is in thermal communication with the volatile material 204 through the wick 356. The controller 276 is configured to control operation of the heater assembly 284 and other electrical components such as the illumination elements 212. The controller 276 includes a voltage or current regulator and may also include a number of other electrical components, including capacitors, resistors, inductors, fuses, diodes, and so forth. In some embodiments, the controller 276 includes a timer, which may allow the controller 276 to shut off specific electronic components for a predetermined amount of time after a predetermined amount of time of use.

Still referring to FIG. 10, the controller 276 includes a plurality of components disposed thereon. The controller 276 includes the wireless communication unit 280 or receiver that is a module that supports wireless communication to the electronic device 216. The controller 276 may further include a first regulator and a second regulator, either of which may be a voltage or current regulator. In some embodiments the wireless communication unit supports Bluetooth® Low Energy (BLE) wireless communication, Wi-Fi, or other types of wireless communication. The wireless communication unit 280 may include one or more of onboard crystal oscillators, chip antenna, and/or passive components. The wireless communication unit 280 may support a number of peripheral functions, e.g., an application deliver controller (“ADC”), timers, counters, pulse width modulation (“PWM”), and serial communication protocols. Some of the serial communication protocols used through the wireless communication system's programmable architecture includes I2C, universal asynchronous receiver/transmitter (“UART”), and serial peripheral interface (“SPI”). The controller 276 may be or include a microcontroller unit (“MCU”) and/or an application-specific integrated circuit (“ASIC”). The controller 276 includes a processor, a flash memory, and additional components not specifically noted herein. The controller 276 may utilize one or more auxiliary inputs 428 that are configured to provide information such as user preferences, switch selection, and environmental factors.

Still referring to FIG. 10, in some embodiments the power supply 424 is configured to receive a USB-C type plug, that can be used to charge or power the dispenser 208. In some embodiments, the dispenser 208 receives power from a wall outlet, a car lighter socket, or another source of power as the power supply 424. In some embodiments the power supply 424 may be a battery which could include a rechargeable battery, a one-time use battery, a lead-acid battery, a nickel-cadmium battery, a nickel-metal hybrid battery, a lithium-ion battery, an alkaline battery, a zinc-carbon battery, a coin cell battery, a zinc-air battery, a sealed lead-acid battery, or any other device known in the art that holds energy in the form of chemicals. In some embodiments, the power supply 424 may use a combination of battery types and/or the power supply 424 may be a combination of power sources such as a battery and a wall charger. In some embodiments, the power supply 424 may implement power conditioning to transform the line voltage to 5V DC. In some embodiments, the power supply 424 may not implement power conditioning.

Still referring to FIG. 10, the heater assembly 284 is in thermal communication with the volatile material 204. In some embodiments, the heater assembly 284 may be a ceramic annular disk with an inlayed potted metal oxide resistor. In some embodiments, the heater assembly 284 comprises ceramic with an inlayed potted metal oxide resistor that is not in an annular shape. Further, the heater assembly 284 may be a tubular metal oxide resistor, a kapton heater, a foil heater, or a copper heating coil. In some embodiments, the heater assembly 284 comprises a honeycomb configuration such that it is considered a honeycomb heater.

Further, the heater assembly 284 is configured to receive the wick 356 that extends upwardly into contact with the heater assembly 284. The heater assembly 284 is configured to receive the wick 356 that extends upwardly without contact with the heater assembly 284. Alternatively, the heater assembly 284 may be a contactless laser LED or a consumable heater assembly such as a tungsten filament. In some embodiments, the heater assembly 284 may be integrated in the refill 228. In some embodiments, the heater assembly 284 comprises a plurality of heater assemblies that surround the wick 356. The plurality of heater assemblies may be arranged in such a way that they are able to sequentially heat the wick 356. In some embodiments, the heater assembly 284 may be a nichrome wire that is embedded in the wick 356 that is attached to and/or along portions of the refill 228. In some embodiments, the heater assembly 284 may be a pin-point heater. In some embodiments, the heater assembly 284 may comprise one or more resistors.

Still referring to FIG. 10, the volatile material 204 is in direct contact with the heater assembly 284 through the wick 356. In some embodiments the wick 356 is not in direct contact with the heater assembly 284. In some embodiments, the refill 228 is in thermal communication with the heater assembly 284 such that the wick 356 brings the volatile material 204 close enough in proximity to heater assembly 284 to facilitate evaporation of the volatile material 204 faster than an evaporation in a controlled setting. In some embodiments, the heater assembly 284 is in communication with the refill 228 via the wick 356 and transports the volatile material 204 from the refill 228 to the heater assembly 284. In some embodiments, the wick 356 may be a sintered wick such as a POREX® Wick. In some embodiments, the fluid delivery system shown as the wick 356 in FIG. 10 is a different type of liquid transfer mechanism. In some embodiments, the fluid delivery system may be a gravity fed injector or an apparatus for storing fluid that has a connection to a drip pipe disposed below the apparatus.

In some embodiments, the fluid delivery system is a solenoid or an electronic fuel injector type dispenser that can periodically deliver a metered amount of volatile material from the refill 228 to the heater assembly 284 for volatilization. The timing and delivery of volatile material 204 can be controlled by the controller 276, which can then provide a precise heating sequence to the heater assembly 284 based upon when volatile material 204 is placed into contact with the heater assembly 284. Regardless of the type of fluid delivery system (e.g., the wick 356), the heater assembly 284 is in thermal communication (e.g., direct contact or indirect contact) with liquid carried by the fluid delivery system. In some embodiments, the wick 356 is a sintered wick to which a spring is coupled to facilitate contact between the wick 356 and the heater assembly 284. The sintered wick with the spring may offer compliant contact to accommodate varying tolerances of components of the dispenser 208. In some embodiments, the heater assembly 284 and the refill 228 may be in communication by the heater assembly 284 extending into a small cylinder cutout within the top of the refill 228.

Still referring to FIG. 10, in some embodiments, the refill 228 is a container for the volatile material 204 that is a liquid. In some embodiments, the refill 228 is a pre-dosed pad or gel. In some embodiments, the refill may be integral and nonremovable from the dispenser 208. In some embodiments, the refill 228 may be a refillable container that includes a cartridge. The cartridge is configured to include additional elements, such as a base that is coupled with the cartridge. In some embodiments, the refill 228 may be a glassomizer, a clearomizer, or a catromizer, or may be another type of device that is used to deliver liquid to vapor. In some embodiments, the refill 228 is configured for one-time use such that the wick 356 and the heater assembly 284 are embedded within the refill 228. In some embodiments, the refill 228 is configured to be used for multiple uses and may also be detachable from the dispenser 208. In some embodiments the refill 228 is configured to be removed and replaced by another refill. In some embodiments, the refill 228 may be an Aspire™ Nautilus tank that implements an adjustable airflow ring at a bottom end thereof that allows for various heat settings. In some embodiments, the refill 228 includes a removable tank, which may comprise Pyrex®.

In some embodiments, the refill 228 holds the volatile material 204 that includes one or more compositions, which may be any suitable liquid or liquids, and includes one or more active ingredients. As noted above, active ingredients include, but are not limited to, one or more of a cleaner, an insecticide, an insect repellent, an insect attractant, a disinfectant, a mold or mildew inhibitor, an antimicrobial, a fragrance comprised of one or more aroma chemicals, a disinfectant, an air purifier, an aromatherapy scent, an antiseptic, an odor eliminator, a positive fragrance active material, an air-freshener, a deodorizer, a medicinal component, an inhalant (e.g., for relieving a cough or congestion), or the like, and combinations thereof. In some embodiments, the dispenser 208 disclosed herein is used as a pest control product that has the ability to operate in both an unfragranced and a fragranced repellent mode (e.g., at a lower temperature setting which outputs a very light or negligible fragrance and at a higher temperature setting which outputs a fragrance).

Still referring to FIG. 10, the dispenser 208 is configured to control the volatile material concentration in the environment by controlling an amount of the volatile material 204 that is evaporated from the refill 228 by modifying a voltage or a current supplied to the heater assembly 284. In some embodiments, the controller 276 is configured to monitor the temperature of heater assembly 284 by monitoring the voltage or current supplied to the heater assembly 284. Still referring to FIG. 10, the volatile material 204 within the refill 228 is in fluid communication with the heater assembly 284, which may be a pin-point heater. By using a heater such as a pinpoint heater, desirable temperatures can be achieved relatively quickly. In some embodiments, the heater assembly 284 may be turned “on” for a few seconds, every few minutes to achieve an activation period. During the activation period, the power level delivered to the heater assembly 284 may be varied (e.g., between a range of power settings) to achieve an average target power setting (e.g., 5.0 W) over the activation period. In other examples, the heater assembly 284 may be activated and held at a constant power level throughout the activation. Through the use of PWM, the heater assembly 284 can be adjusted to a range of different temperatures, and by achieving varying temperatures, different volatile material evaporation rates can be produced and output.

Since small deviations in temperature may cause different volatile material concentrations or different scent notes in the environment that are perceptible by a user, some embodiments of the present disclosure implement the auxiliary inputs 428 that includes discrete user inputs through settings that provide for different intensities that are perceptible by a user, e.g., “low,” “medium,” and “high.” In some embodiments, 4.0 Watts (W) of power may be associated with a “low” setting, 5.0 W of power may be associated with a “medium” setting, and 6.0 W of power may be associated with a “high” setting.

Still referring to FIG. 10, in some embodiments, the dispenser 208 is configured to move the heater assembly 284 closer to or farther from the volatile material 204 depending on an intensity selection of the user and/or other factors including environmental factors and presence of a child or pet. In such embodiments, the dispenser 208 is configured to move the heater assembly 284 closer to the refill 228 to increase the intensity of the heat transferred from the heater assembly 284 to the refill 228. Thus, the amount of the volatile material 204 evaporated into the environment may increase as a result of an increased rate in energy transfer from the heater assembly 284 to the volatile material 204 contained within the refill 228. In some embodiments, the dispenser 208 is configured to move the heater assembly 284 away from the refill 228 to decrease the amount and intensity of the energy transferred from the heater assembly 284 to the refill 228. Thus, the amount and intensity of the volatile material released into the environment may decrease. In some embodiments, the dispenser 208 is configured to control the rate of volatile material evaporation by moving the heater assembly 284 along the refill 228, which may comprise a pre-dosed pad or gel, to continuously expose the heater assembly 284 to a new area or region of the volatile material composition. In some embodiments, the heater assembly 284 may be moved using a pendulum or another type of oscillating device.

Still referring to FIG. 10, the flow rate of the volatile material 204 from the refill 228 to the heater assembly 284 through the wick 356 defines an amount of the volatile material 204 that is evaporated and released into the environment. In some embodiments, the flow rate of volatile material 204 from the refill 228 to the heater assembly 284 is controlled using a secondary heater assembly (not shown) to heat or cool the refill 228. Heating the refill 228 with the secondary heater assembly (not shown) may decrease the viscosity of the volatile material 204, thereby increasing the flow from the refill 228 to the heater assembly 284 via the wick 356. In a similar fashion, the secondary heater assembly (not shown) may be turned off or the power output may be lowered to increase the viscosity of the volatile material 204 in the refill 228 to reduce the flow rate of the volatile material 204 from the refill 228 to the heater assembly 284 via the wick 356.

In some embodiments, the dispenser 208 uses duty cycles to achieve the desired volatile material concentration in the surrounding environment. The dispenser 208 is configured to have a power switch that is used to power “on” and “off” the dispenser 208. In some embodiments, the switch is electrical and controlled by the controller 276. When the power switch is activated to an “on” position, the controller 276 directs electric current to flow to the heater assembly 284 using power supplied by the power supply 424. Once a target temperature is achieved, the controller 276 can shut off the electrical current supplied to the heater assembly 284. In some embodiments, a PWM algorithm is used to allow the heater assembly 284 to heat up quickly, which in turn may allow a faster fragrance or volatile material release.

Still referring to FIG. 10, the controller 276 is configured to receive information from the auxiliary inputs 428 which includes environmental sensors, user inputs, and/or information from a lookup table regarding chemical characteristics from a set of volatile material characteristics. In some embodiments, the environmental sensors may comprise the sensors listed below that may detect the cartridge or the type of volatile material within the cartridge. The controller 276 includes a processor that processes the information to determine an optimal power level for an optimal heating temperature and heating duration to produce a desired volatile material concentration.

Still referring to FIG. 10, in some embodiments, the environmental sensors that are used include, but are not limited to, a microphone, a camera, a turbidity sensor, a thermometer, a humidity sensor, a passive infrared sensor, a light sensor, a lightning sensor, a wind transducer, a compass, a Global Positioning System (“GPS”), a gyroscope, an accelerometer, a barometer, a crash sensor, a proximity sensor, a radar, an ultrasonic sensor, or any combination thereof. In some embodiments, the environmental sensors are located on the dispenser 208. In some embodiments, the environmental sensors are located on the electronic device 216. In some embodiments, the collected data from the environmental sensors is transferred from the electronic device 216 to the dispenser 208. In some embodiments, the electronic device 216 collects data from software applications that hold data regarding environmental sensors external to the electronic device 216. The electronic device 216 receives the harvested data from the external environmental sensors and either communicates the data to the dispenser 208 or communicates a command to the dispenser 208 based on the data from the external environmental sensors.

Still referring to FIG. 10, in some embodiments, the chemical characteristics of the volatile material 204 are measured by the dispenser 208 or transmitted to the controller 276 through a wireless communication device or other means. Some of the user inputs include but are not limited to time preferences for operation, intensity level, and child mode. In some embodiments, the controller 276 receives the information from the environmental sensor(s), the user inputs, and the chemical characteristics of the volatile material, and regulates the dispenser 208 to achieve a desired heating temperature of the heater assembly. Thus, the temperature correlates with a desired volatile material concentration level in the surrounding environment.

Still referring to FIG. 10, in some embodiments, the auxiliary inputs 428 includes user inputs which are input into the dispenser 208 via external switches. In some embodiments, a first switch is configured to control an intensity of the fragrance. A first switch setting defines a “low” setting, which may set the heater assembly 284 to operate with 5 minutes between each operational cycle. A second switch setting defines a “medium” setting that may set the heater assembly 284 to operate with 3 minutes between each operational cycle. A third switch setting defines a “high” setting that may set the heater assembly 284 to operate with one minute between each operational cycle. By adjusting the first switch between the three or more switch settings, the timing between operational cycles differs so that different fragrance intensities and volatile material intensities can be achieved in the surrounding environment. In some embodiments, there may be more than one switch. In some embodiments, the first switch and the second switch may additionally or alternatively be provided via a display screen of the electronic device 216 that is in communication with one or more of the electrical components within the dispenser 208. A user may interact with the electronic device 216 to adjust fragrance intensities and volatile material concentrations.

In some embodiments, the dispenser 208 includes features that provide enhanced customization and adaptation capabilities. Algorithms may be used by the dispenser 208 to modify the operational parameters according to user preferences and/or volatile material requirements. Generally, the controller 276 operates the volatile material dispenser 208 according to pre-programmed sequences, which are designed to control the temperature of the heater assembly within 1° F. (0.55° C.) of the targeted temperature. More specifically, algorithms may be used to vary the power applied to the heater. For example, PWM may be used to adjust the heater assembly 284 to different temperatures according to various duty cycles.

In some embodiments, controlling the temperature of the heater assembly 284 results in controlling evaporated volatile material intensities and fragrance characteristics of the evaporated volatile materials, which creates a consistent volatile material intensity and fragrance experience despite the particular user environment. The operation of the dispenser 208 is adjusted directly through user manipulated controls or wirelessly through the electronic device 216, such as a user's mobile device. In some embodiments, the heater assembly 284 is configured to achieve a temperature of between about 100° F. and about 300° F., or between about 125° F. and about 275° F., or between about 150° F. and about 250° F., or between about 175° F. and about 225° F. The heater assembly 284 may comprise one or more resistors having a resistance of between about 0.5 Ohms (Ω) and about 50.0Ω, or between about 1.0Ω and about 15.0Ω, or between about 1.5Ω and about 10.0Ω, or between about 2.5Ω and about 7.5Ω, or between about 3.5Ω and about 5.0Ω.

In some embodiments, the power limits vary for different volatile materials or different environments. Further, the intensity limits (e.g., on time/off time between activations) are also different among different volatile materials and environments. In some embodiments, both duration and intensity limits are changed throughout operation of the dispenser 208 due to changes that may occur over time and/or due to one or more of a number of external factors that are measured or identified either automatically or from information input by a user. In some embodiments, automatic triggers cause the duration limits or intensity limits (e.g., minimum and maximum switch positions) to be adjusted, for example, based on a time of the day, a measured room temperature, a size of the room, a season of the year, the weather, or vital signs of a person acquired from the electronic device (e.g., a cell phone, a smart watch, or another type of device having sensors or that can receive user inputs). The changes in intensity levels are also triggered via request from an app or via direct physical interaction with the dispenser 208.

In some embodiments, the volatile material characteristics are enhanced through different durations of operation and/or different frequencies of operation. For example, different frequencies of operation could include the dispenser 208 operating a duty cycle every 5 minutes or every 15 minutes. In some embodiments, the volatile material characteristics are enhanced through an algorithm that varies the temperature of activation in a predetermined fashion and/or through the use of an on/off timer. In some embodiments, a user is capable of controlling the various parameters discussed above through an app on the electronic device 216, which allows a user to vary characteristics of the volatile material by choosing different pre-determined operational points that are connected to volatile material characteristics such as a fragrance or insect repellant. In some embodiments, the operational points may be pre-determined or pre-programmed.

In some embodiments, the refill 228 includes the body 336 having embedded information (e.g., along an outer side thereof) which is automatically detected via one or more sensors within the volatile material sensor. In some embodiments, the dispenser 208 includes one or more sensors, such as an infrared (IR) sensor, an optical sensor, a weight sensor, a hall effect sensor, one or more magnets, a radio frequency identification (RFID) sensor, a barcode scanner, a QR scanner, a proximity sensor, a reflective photo interpreter, a humidity sensor, a fluid property sensor, a light sensor, an alcohol sensor, or another type of sensor. In some embodiments, the dispenser 208 is configured to retrieve data that provides information regarding the collected sensory input from the sensors. For example, the dispenser 208 is configured to read a QR code (e.g., via a camera) that may scan a QR code along the body 336. The QR code may be associated with embedded information including anticipated humidity level, pressure levels, temperature, or any other type of information that may influence the timing of the run cycle(s) and the heating intensity of the heater assembly 284. In some embodiments, the volatile material 204 may be produced for or configured for volatilization in a tropical environment. The dispenser 208 may then vary the temperature of the heater assembly 284 based on receiving information from the refill 228 that the volatile material 204 is likely in a particular environment such as a tropical environment.

In some embodiments, data from one or more of the above-referenced sensors is received by the controller 276. The controller 276 is configured to have a memory, a processor, and information stored within a lookup table that can be compared against data that is retrieved by any of the sensors. An identified fragrance or fragrance characteristic of the volatile material 204 is determinable based on the comparison of the data with the information stored within the lookup table. However, in some embodiments a user manually inputs information that identifies a refill type or the environmental conditions. In some embodiments, the refill 228 is a cartridge that includes two volatile materials that are configured to release two different volatile material characteristics, i.e., the cartridge is a 2-tone cartridge. In some embodiments, the volatile material may be identified based on data that is retrieved by the one or more sensors noted above in combination with data manually input by a user.

In some embodiments, a lock-and-key feature is provided with the refill 228 in the form of a cartridge. For example, the lock-and-key feature may be a protrusion along an upper rim of the refill 228, which may provide information to the dispenser 208 regarding the volatile material within the refill 228. The lock-and-key feature may be disposed along the body 336 or may be provided along an inner surface of the body 336. The dispenser 208 is configured to detect a type of volatile material 204 that is within the refill 228 based on a location of the lock-and-key feature. For example, a refill filled with a volatile material that works well in the tropics may be identified based on a location of two radially offset protrusions along an exterior surface of the cartridge.

In some embodiments, the sensor measurements and temperature measurements are associated with different, pre-determined wattages, which can be pre-determined based on an identified vapor pressure of each of the particular volatile materials to be emanated from the dispenser 208 that corresponds with a temperature that is achieved based upon a specified power setting, e.g., 5.0 W, 5.5 W, and 6.0 W. Although ranges of 5.0 to 6.0 W are discussed, various other heating profiles are possible (e.g., between 1.0 W and 9.0 W) and can be chosen based upon the volatile material present within the refill 228.

Still referring to FIG. 10, in some embodiments, a network 432 is in communication with the electronic device 216 and the dispenser 208. The electronic devices 216 that are in communication with the network 432 include but are not limited to a Google® Nest® Thermostat, a home security system, an Amazon® Alexa®, a Ring® Video Doorbell, a motion detector, a garage door opener, a gate access opener, a neighborhood guardhouse, a smart T.V., a thermostat, an automatic lock, an HVAC unit, and/or any other smart home device. In some embodiments, the electronic device 216 includes a wireless communication device such as a mobile phone with a graphical user interface (GUI), or any other device, such as, e.g., a laptop, a tablet, a desktop, a server, or a special purpose computer. In some embodiments, the dispenser 208 may be in communication with more than one of the electronic devices 216.

The network 432 is any suitable network or combination of networks. For example, the network 432 includes a Wi-Fi network (which can include one or more wireless routers, one or more switches, etc.), a peer-to-peer network (e.g., a Bluetooth® network), a cellular network (e.g., a 3G network, a 4G network, a 5G network, etc., complying with any suitable standard, such as CDMA, GSM, LTE, LTE Advanced, NR, etc.), a wired network, etc. In some embodiments, the network 432 can be a local area network (LAN), a wide area network (WAN), a public network (e.g., the Internet), a private or semi-private network (e.g., a corporate or university intranet), any other suitable type of network, or any suitable combination of networks.

Still referring to FIG. 10, the communications network systems (not shown) that are used with the network 432 include any suitable hardware, firmware, and/or software for communicating information over the network 432 and/or any other suitable networks 432. For example, the network systems (not shown) can include one or more transceivers, one or more communication chips and/or chip sets, etc. In a more particular example, the network systems (not shown) include hardware, firmware and/or software that are used to establish a Wi-Fi connection, a Bluetooth® connection, a cellular connection, an Ethernet connection, etc.

Still referring to FIG. 10, in some embodiments, the user scans a QR code on the side of the refill 228 using the electronic device 216 such as a mobile phone. The electronic device 216 then sends information pertaining to the optimal power output cycles to the dispenser 208 from data stored on the QR code using BLE or a Wi-Fi network. In some embodiments, the electronic device 216 sends information to the dispenser 208 when the user either selects a volatile material type from a drop-down list in a software application or inputs a numerical in the software application on the electronic device 216. The information input from the user is then sent to the controller 276 to determine an optimal power output cycle or the electronic device 216 may use the information to determine the optimal power output cycle and send the optimal power output cycle to the controller 276.

Still referring to FIG. 10, in some embodiments, the dispenser 208 is configured to receive information related to the ambient temperature received from the electronic device 216, which may be a thermostat, a phone, or other electronic device, and the controller 276 is configured to adjust the power output to the heater assembly 284 based on the ambient temperature received from the electronic device 216. Further, the controller 276 is configured to only apply the ambient temperature from the electronic device 216 and may not take into account the temperature of the refill 228 and/or the temperature of the heater assembly 284 when determining and outputting the output power to the heater assembly 284.

Referring now to FIG. 11, an example graphical user interface 436 (“GUI”) allows the user to input commands, causing the electronic device 216 to send the commands from the electronic device 216 to the dispenser 208. The GUI 436 is an interactive software application, downloadable to the electronic device 216. The GUI 436 includes a plurality of soft buttons 440. The example GUI 436 may be presented to the user when the user opens the software application on the electronic device 216. The soft buttons 440 allow the user to perform functions such as turning the dispenser 208 on and off, setting a release rate of the dispenser, setting a dispensation schedule, or controlling the brightness or color of the plurality of illumination elements 212. The GUI 436 provides the user an indication of the status of an input. In some embodiments, the GUI 436 provides a first visual status indicator (such as a spinning wheel) when the user inputs a command. The GUI 436 then provides a second visual status indicator to communicate that the command is completed.

In some embodiments, the GUI 436 offers the option of an instant boost 442. Selecting the instant boost 442 causes the dispenser 208 to dispense a relatively larger volume of the volatile material 204 in response to the command. In some embodiments, the instant boost 442 is selected when the user is overwhelmed by a powerful stench or a large number of pests.

In some embodiments, the GUI 436 provides the user an indication of the amount of volatile material 204 remaining in the refill 228. In some embodiments, the dispenser 208 calculates the amount of volatile material 204 remaining in the refill 228 by tracking a temperature of the heater assembly 284 and a duration that the heater assembly 284 is heating the volatile material 204. Using the temperature of the heater assembly 284 and the duration that the heater assembly 284 has heated the volatile material 204, the dispenser 208 estimates a volume of volatile material emanated from the dispenser 208. The dispenser 208 then communicates an estimate of the amount of volatile material remaining in the refill 228 to the electronic device 216 in the form of a percentage of the total volatile material remaining, or in the form of a volume unit such as milliliters or fluid ounces that remain within the refill 228. In some embodiments, the software application notifies the electronic device 216 when a predetermined amount of the volatile material 204 remains in the refill 228. In some embodiments, the software application includes the soft button 440 configured to open an online marketplace for the user to purchase extras of the refills 228.

In some embodiments, the dispenser 208 is configured to sense when the electronic device 216 is wirelessly connected to the dispenser 208. In some embodiments, the dispenser 208 is configured to heat the volatile material 204 using the heater assembly 284 when the electronic device 216 is wirelessly connected to the dispenser 208. In some embodiments, the dispenser 208 is configured to sense how far the electronic device 216 is from the dispenser 208. The dispenser 208 senses that the electronic device is in a predetermined radius and activates at least one of the illumination elements 212 or the heater assembly 284. In some embodiments, the connection of the electronic device 216 to the dispenser 208 does not trigger the illumination elements 212 or the heater assembly 284.

Referring to FIG. 12, schematic representations of an illumination sequence of the light ring 240 are shown. The front wall 232 of the housing includes the plurality of illumination elements 212, illustrated as light emitting diodes (LEDs). The example illumination elements 212 are configured to illuminate the light ring 240 in the front wall 232. In some embodiments, the illumination elements 212 act as a dispenser GUI 444, communicating the status of the dispenser 208 to the user. In the example illustrated in FIG. 12, a first illumination element 448 is illuminated at a two o'clock position 452 illuminating a first light ring region 456, then a second illumination element 460 is illuminated at a six o'clock position 464 illuminating a second light ring region 468, and then a third illumination element 472 is illuminated at a ten o'clock position 476 illuminating a third light ring region 480, in a chasing pattern.

To clarify, the present example shows the first illumination element 448 illuminated, then when the second illumination element 460 is illuminated, the first illumination element 448 is no longer illuminated, and then when the third illumination element 472 is illuminated, the second illumination element 460 is no longer illuminated. In some embodiments, the illumination elements 212 are arranged in an equilateral triangular configuration. In some embodiments, the chasing pattern continuously revolves around the light ring 240, while the dispenser 208 attempts to connect to the electronic device 216 to the network 432. In some embodiments, when a successful connection is established between the dispenser 208 and the electronic device 216, the first, second, and third light ring regions 456, 468, 480 are illuminated. In some embodiments, the first, second, and third light ring regions 456, 468, 480 are discrete. In some embodiments, first, second, and third light ring regions 456, 468, 480 overlap.

Still referring to FIG. 12, the dispenser GUI 444 includes the emblem 244. As noted above, the emblem 244 is capable of being illuminated to indicate a status of the dispenser 208. In some embodiments, the emblem 244 lights up to indicate that the dispenser 208 is actively emanating volatile material 204. In some embodiments, when the dispenser 208 is emanating volatile material 204, the light ring 240 indicates a rate of release of the volatile material 204. In order to show the rate of release, one or more of the first, second, and third light ring regions 456, 468, 480 are illuminated. For example, when the dispenser 208 is emanating the volatile material 204 at a standard rate, the first light ring region 456 and the second light ring region 468 illuminate to communicate to the user the rate of dispensation. In another example, when the dispenser 208 is emanating the volatile material 204 at a high rate, each of the first, second, and third light ring regions 456, 468, 480 are illuminated.

In some embodiments, the illumination of any combination of the first, second, and third light ring regions 456, 468, 480 indicate various levels of dispensation, and statuses of the dispenser 208. The first, second, and third light ring regions 456, 468, 480 may also be illuminated with different colors to indicate various levels of dispensation, and statuses of the dispenser 208. For example, when the dispenser 208 is in a child or pet mode the dispenser 208 is configured to illuminate one or more of the first, second, and third light ring regions 456, 468, 480 green, or any other color (e.g., blue, yellow, red, purple, orange, etc.). The first, second, and third light ring regions 456, 468, 480 may also illuminate and/or change color when the refill 228 is empty.

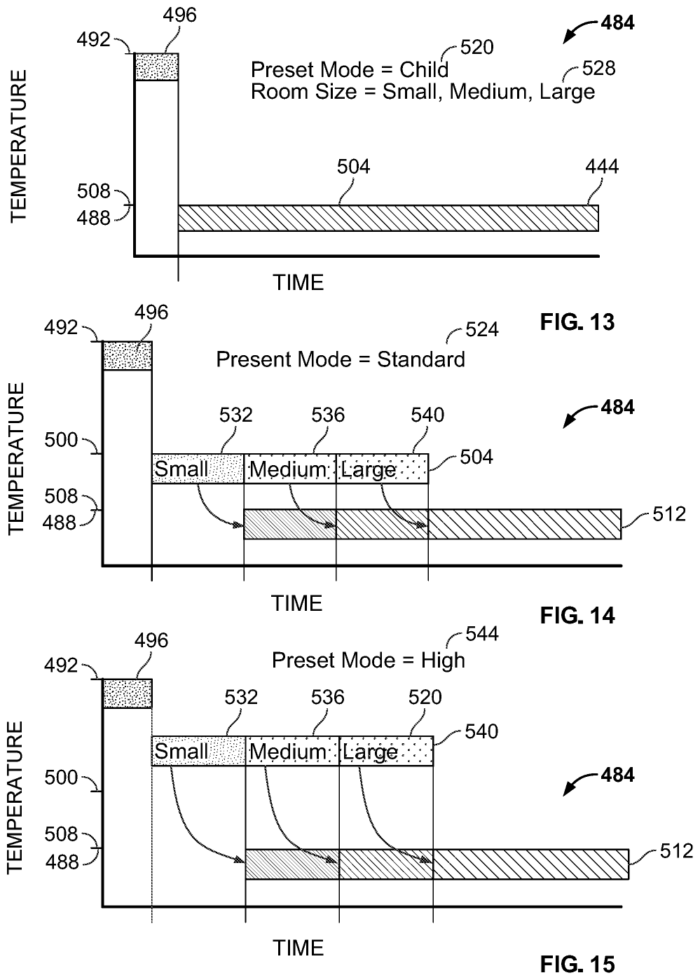

Referring to FIG. 13, the dispenser 208 is configured to release the volatile material 204 at different rates depending on the selected heating profile. FIG. 13 depicts an example of a heating profile 484 of the heater assembly 284 for a preset mode. The heating profile 484 is shown comparing temperature on the vertical axis and time on the horizontal time axis. The heating profile 484 includes one or more temperature levels 488 that are used to heat the volatile material 204. The temperature of the temperature level 488 increases as the temperature level 488 is moved vertically upward on the temperature axis. The heating profile 484 of the preset mode is configured to heat the volatile material 204 at a first temperature level 492 for a first amount of time 496. In some embodiments, the heating profile 484 is then configured to heat the volatile material 204 at a second temperature level 500 for a second amount of time 504. The heating profile 484 includes temperatures of between about 100° F. (38° C.) and about 300° F. (149° C.), or between about 125° F. (52° C.) and about 275° F. (135° C.), or between about 150° F. (66° C.) and about 250° F. (121° C.), or between about 175° F. (79° C.) and about 225° F. (107° C.). The duration of the first amount of time 496 may be between about 0 minutes and 120 minutes, or between about 0 minutes and 90 minutes, or between about 0 minutes and 60 minutes, or between about 0 minutes and 30 minutes, or between about 0 minutes and 20 minutes, or between about 0 minutes and 15 minutes, or between about 0 minutes and 10 minutes, or between about 0 minutes and 5 minutes, or between about 0 minutes and 1 minute.

FIG. 13 depicts the example heating profile 484 for the dispenser 208 that is in a “child mode” 520. The child mode 520 includes the heating profile 484 with the first temperature level 492 heating the volatile material 204 for the first amount of time 496, and the second temperature level 500, having a temperature that is lower than the first temperature level 492, heating the volatile material 204 for the second amount of time 504.

Referring to FIG. 14, the dispenser 208 is also configured to release the volatile material 204 at different rates and in different modes. FIG. 14 depicts an example of the heating profile 484 of the heater assembly 284 for a preset mode that is a “standard mode” 524. The heating profile 484 is configured to heat the volatile material 204 at the first temperature level 492 for the first amount of time 496. In some embodiments, the heating profile 484 is then configured to heat the volatile material 204 at the second temperature level 500 for the second amount of time 504. In some embodiments, the heating profile 484 is then configured to heat the volatile material 204 at a third temperature level 508 for a third amount of time 512.

In some embodiments, the heating profile 484 of the standard mode 524 is dependent upon a room size 528 where the dispenser 208 is located. The present example depicts the heating profile 484 of the dispenser 208 located in a small room 532, in a medium room 536, and in a large room 540. In some embodiments, using the GUI 436 specified above, the user selects the room size 528 where the dispenser 208 is located. The standard mode 524 includes the first temperature level 492, the second temperature level 500, and the third temperature level 508. In some embodiments, in standard mode, the dispenser 208 heats the volatile material 204 at the first temperature level 492 for the first amount of time 496. The dispenser 208 then heats the volatile material 204 at the second temperature level 500 for the second amount of time 504. As depicted in FIG. 14, the room size 528 influences a duration of the second amount of time 504. For example, the dispenser 208 located in the large room 540 is depicted to heat the volatile material 204 at the second temperature level 500 for the second amount of time 504, which is longer than the second amount of time 504 that the dispenser 208 located in the small room 532 is configured to heat the volatile material 204 at the second temperature level 500. In some embodiments, the increase of duration of the second amount of time 504 for the dispenser 208 located in the large room 540 allows for greater amounts of the volatile material 204 to be released to spread throughout the large room 540.

FIG. 15 depicts the heating profile 484 that is configured to heat the volatile material 204 at three separate temperature levels for three separate durations of time in a “high mode” 544. In some embodiments, the heating profile 484 of the high mode 544, like the standard mode 524, is dependent upon the room size 528 where the dispenser 208 is located. In some embodiments, the heating profile 484 of the high mode 544 is substantially similar to the heating profile 484 of the standard mode 524; however, the temperature of the first temperature level 492, the second temperature level 500, or the third temperature level 508 of the high mode 544, is higher than the temperature of the first temperature level 492, the second temperature level 500, or the third temperature level 508 of the standard mode 524. In some embodiments, only the temperature of the second temperature level 500 of the high mode 544 is higher than the temperature of the second temperature level 500 of the standard mode 524.

Though FIGS. 14 and 15 illustrate a maximum of three temperature levels and three amounts of time, the heating profile 484 may include any number of heating levels and any number of amounts of time. In some embodiments, the heating profile 484 oscillates between the first temperature level 492 and the second temperature level 500, or the second temperature level 500 and the third temperature level 508, and so on. Though the present example shows that the first temperature level 492 has a temperature that is higher than the temperature of the second temperature level 500, in some embodiments, the first heating level 492 has the temperature that is lower than the temperature of the second temperature level 500.

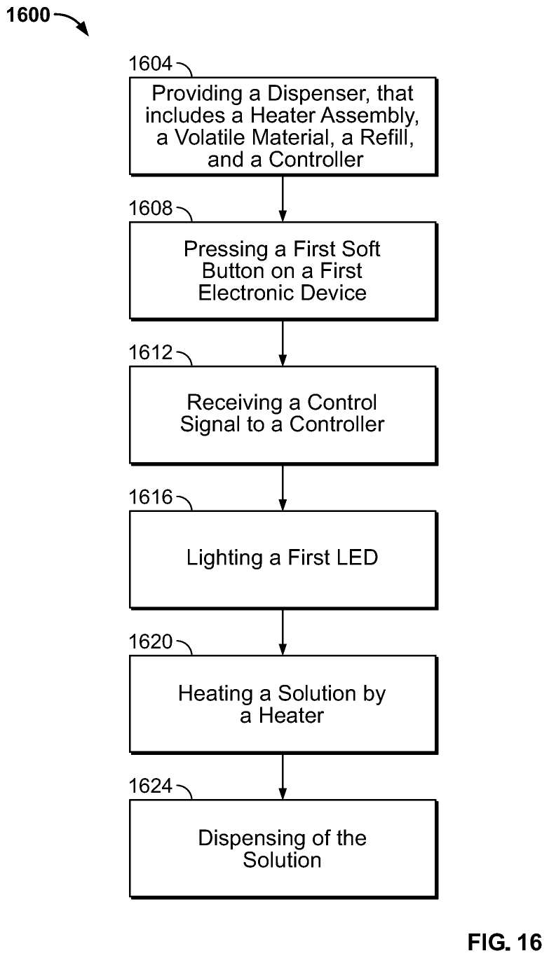

Referring now to FIG. 16, a method 1600 is illustrated for emitting a volatile material, which may include fewer or more steps than depicted. In some embodiments, the following steps are performed in any order. At a first step 1604, the method 1600 includes providing a volatile material dispenser, the volatile material dispenser including a heater assembly, a volatile material, a refill, and a controller. At a second step 1608, the method 1600 includes pressing a first soft button on a first electronic device. At a third step 1612, the method 1600 includes receiving a control signal to the controller on the dispenser. At a fourth step 1616, the method 1600 includes lighting a first illumination element, where the illumination element is an LED. At a fifth step 1620, the method 1600 includes heating the volatile material by the heater. At a sixth step 1624, the method 1600 includes dispensing of the solution.

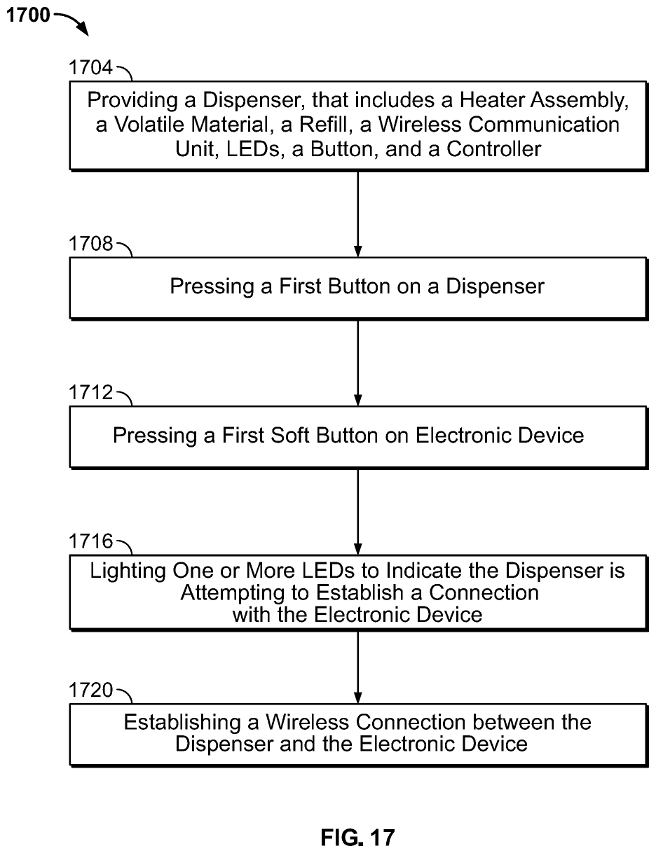

Referring now to FIG. 17, a method 1700 is illustrated for emitting a volatile material, which may include fewer or more steps than depicted. In some embodiments, the following steps are performed in any order. At a first step 1704, the method 1700 includes providing a volatile material dispenser, the volatile material dispenser including a heater assembly, a volatile material, a refill, LEDs, a wireless connection unit, a button, and a controller. At a second step 1708, the method 1700 includes pressing the button on the dispenser. At a third step 1712, the method 1700 includes pressing a first soft button on an electronic device. At a fourth step 1716, the method 1700 includes lighting one or more of the LEDs to indicate that the dispenser is attempting to establish a connection with the electronic device. At a fifth step 1720, the method 1700 includes establishing a wireless connection between the dispenser and the electronic device.

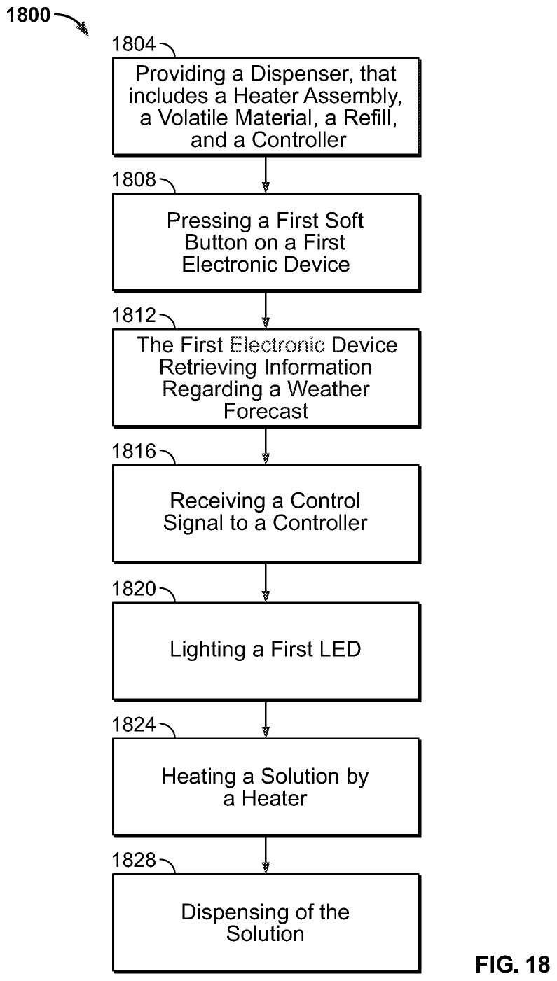

Referring now to FIG. 18, a method 1800 is illustrated for emitting a volatile material, which may include fewer or more steps than depicted. In some embodiments, the following steps are performed in any order. At a first step 1804, the method 1800 includes providing a volatile material dispenser, the volatile material dispenser including a heater assembly, a volatile material, a refill, and a controller. At a second step 1808, the method 1800 includes pressing a first soft button on a first electronic device. At a third step 1812, the method 1800 includes the first electronic device retrieving information regarding a weather forecast. At a fourth step 1816, the method 1800 includes receiving a control signal to the controller on a dispenser. At a fifth step 1820, the method 1800 includes lighting a first LED. At a sixth step 1824, the method 1800 includes heating the volatile material by the heater. At a sixth step 1828, the method 1800 includes dispensing the volatile material.

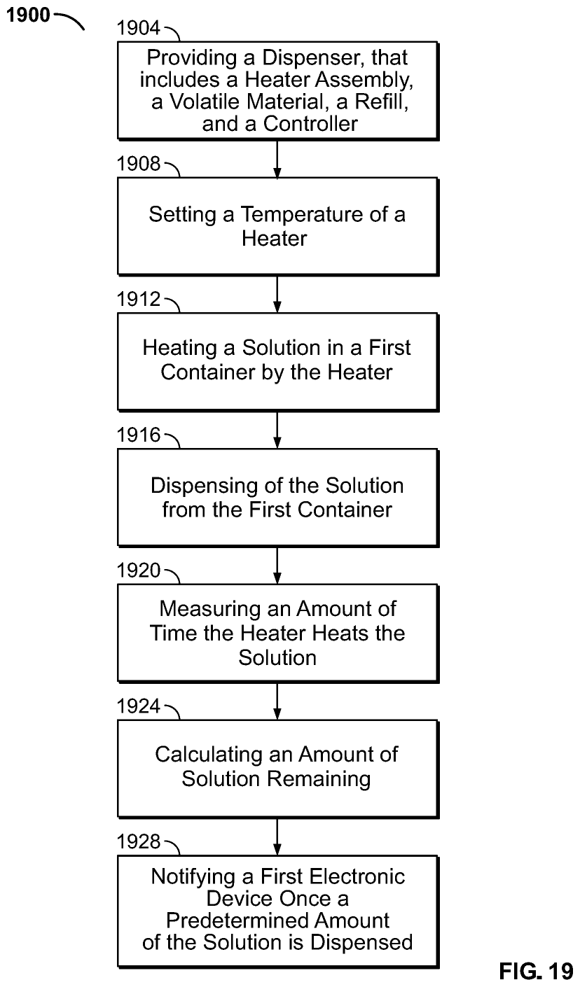

Referring now to FIG. 19, a method 1900 is illustrated for emitting a volatile material, which may include fewer or more steps than depicted. In some embodiments, the following steps are performed in any order. At a first step 1904, the method 1900 includes providing a volatile material dispenser, the volatile material dispenser including a heater assembly, a volatile material, a refill, and a controller. At a second step 1908, the method 1900 includes setting a temperature of the heater. At a third step 1912, the method 1900 includes heating the volatile material in the refill by the heater. At a fourth step 1916, the method 1900 includes dispensing of the volatile material from the refill. At a fifth step 1920, the method 1900 includes measuring an amount of time the heater heats the volatile material. At a sixth step 1924, the method 1900 includes calculating an amount of volatile material remaining. At a seventh step 1928, the method 1900 includes notifying a first electronic device once a predetermined amount of the volatile material is dispensed.

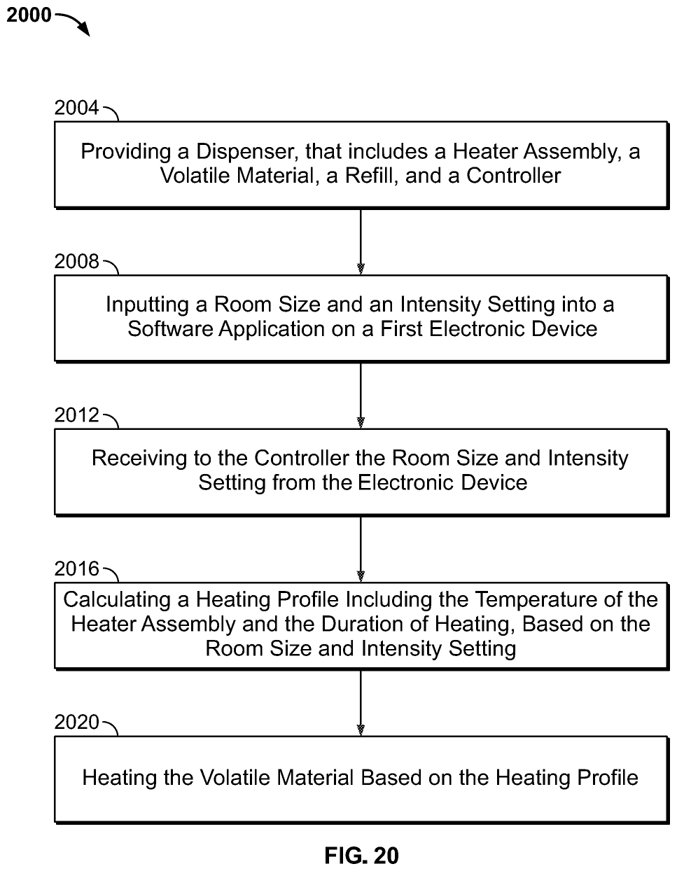

Referring now to FIG. 20, a method 2000 is illustrated for emitting a volatile material, which may include fewer or more steps than depicted. In some embodiments, the following steps are performed in any order. At a first step 2004, the method 2000 includes providing a volatile material dispenser, the volatile material dispenser including a heater assembly, a volatile material, a refill, and a controller. At a second step 2008, the method 2000 includes a user inputting a room size and an intensity setting into a software application on a first electronic device. At a third step 2012, the method 2000 includes calculating a heating profile including the temperature of the heater assembly and the duration of heating, based on the room size and intensity setting. At a fourth step 2016, the method 2000 includes the controller receives a heating profile from the electronic device based on the room size and intensity setting. At a fifth step 2020, the method 2000 includes heating the volatile material based on the heating profile.

Also as used herein, unless otherwise specified or limited, directional terms are presented only with regard to the particular embodiment and perspective described. For example, reference to features or directions as “horizontal,” “vertical,” “front,” “rear,” “left,” “right,” “upper,” “lower,” and so on are generally made with reference to a particular figure or example and are not necessarily indicative of an absolute orientation or direction. However, relative directional terms for a particular embodiment may generally apply to alternative orientations of that embodiment. For example, “front” and “rear” directions or features (or “right” and “left” directions or features, and so on) may be generally understood to indicate relatively opposite directions or features for a particular embodiment, regardless of the absolute orientation of the embodiment (or relative orientation relative to environmental structures). “Lateral” and derivatives thereof generally indicate directions that are generally perpendicular to a vertical direction for a relevant reference frame.

Also as used herein, ordinal numbers are used for convenience of presentation only and are generally presented in an order that corresponds to the order in which particular features are introduced in the relevant discussion. Accordingly, for example, a “first” feature may not necessarily have any required structural or sequential relationship to a “second” feature, and so on. Further, similar features may be referred to in different portions of the discussion by different ordinal numbers. For example, a particular feature may be referred to in some discussion as a “first” feature, while a similar or substantially identical feature may be referred to in other discussion as a “third” feature, and so on.

As used herein, unless otherwise limited or specified, “substantially identical” refers to two or more components or systems that are manufactured or used according to the same process and specification, with variation between the components or systems that are within the limitations of acceptable tolerances for the relevant process and specification. For example, two components can be considered to be substantially identical if the components are manufactured according to the same standardized manufacturing steps, with the same materials, and within the same acceptable dimensional tolerances (e.g., as specified for a particular process or product).

Also as used herein, unless otherwise limited or defined, “integral” and derivatives thereof (e.g., “integrally”) describe elements that are manufactured as a single piece without fasteners, adhesive, or the like to secure separate components together. For example, an element stamped, cast, or otherwise molded as a single-piece component from a single piece of sheet metal or using a single mold, without rivets, screws, or adhesive to hold separately formed pieces together is an integral (and integrally formed) element. In contrast, an element formed from multiple pieces that are separately formed initially then later connected together, is not an integral (or integrally formed) element.

Any of the embodiments described herein may be modified to include any of the structures or methodologies disclosed in connection with different embodiments. Further, the present disclosure is not limited to the dispensing systems of the type specifically shown. Still further, the methods and systems of any of the embodiments disclosed herein may be modified to work with any type of volatile material dispenser.

### INDUSTRIAL APPLICABILITY

Numerous modifications to the present disclosure will be apparent to those skilled in the art in view of the foregoing description. Accordingly, this description is to be construed as illustrative only and is presented for the purpose of enabling those skilled in the art to make and use aspects of the disclosure. The exclusive rights to all modifications which come within the scope of the appended claims are reserved.

We claim:

1. A dispensing system, comprising:
a dispenser having a front portion that is configured to illuminate one or more illumination elements and a rear portion from which a plug deck extends, the rear portion defining a receiving port that is configured to receive a refill,
a heater assembly that is disposed within the rear portion and configured to heat a volatile material disposed in the refill; and
a printed circuit board having a controller and being electrically coupled with the heater assembly, the controller configured to:
control a temperature of the heater assembly,
receive instruction via wireless communication from an electronic device, and
communicate an amount of time that the refill has been heated by the heater assembly to the electronic device,
wherein the one or more illumination elements are configured to communicate a status of the dispenser to a user based on an operational status of the dispenser.

2. The dispensing system of claim 1, wherein the illumination elements are light emitting diodes (LEDs).

3. The dispensing system of claim 1, wherein the front portion includes a light ring disposed along or within the front portion that comprises at least one illumination region.

4. The dispensing system of claim 3, wherein the light ring includes at least three illumination regions configured to illuminate in a pattern to communicate the status of the dispenser.

5. The dispensing system of claim 1, wherein the rear portion includes an illumination region configured to illuminate a surface behind the dispenser.

6. The dispensing system of claim 1, wherein the controller calculates an amount of the volatile material in the refill based on the amount of time that the refill has been heated by the heater.

7. The dispensing system of claim 6, wherein the controller is configured to notify the dispenser when a predetermined amount of volatile material remains in the refill.

8. The dispensing system of claim 1, wherein the volatile material is one of at least a pest repellant or fragrance.

9. The dispensing system of claim 1, wherein at least one of a geo-location, a time of day, a season, or a weather forecast is used to determine a temperature of the heater.

10. A dispenser, comprising:
a front portion that is configured to illuminate three or more illumination elements that are radially offset from and centered about a point;
a rear portion from which a plug deck extends, the rear portion defining a receiving port that is configured to receive a refill,
a heater assembly that is disposed within the rear portion and is configured to heat a volatile material disposed in the refill; and
a printed circuit board having a controller and being electrically coupled with the heater assembly, the controller configured to regulate a temperature of the heater assembly,
wherein the three or more illumination elements, in combination, are configured to communicate a status of the dispenser to a user at least by varying an illumination status of the illumination elements.

11. The dispenser of claim 10, wherein varying the illumination elements includes:
illuminating the illumination elements in a pattern; or
altering an illumination color of the illumination elements.

12. The dispenser of claim 10, wherein the illumination elements are arranged in an equilateral triangular configuration.

13. The dispenser of claim 10, wherein the front portion includes an emblem disposed thereon configured to illuminate to communicate the operational status of the dispenser to the user.

14. A dispenser, comprising:
a front portion that is configured to transmit light emitted by one or more illumination elements;
a rear portion from which a plug deck extends, the rear portion defining a receiving port that is configured to receive a refill,
a heater assembly that is disposed within the rear portion and configured to heat a volatile material disposed in the refill; and
a printed circuit board having a controller and being electrically coupled with the heater assembly, the controller configured to regulate a temperature of the heater assembly, and receive instructions via wireless communication from an electronic device,
wherein the refill is not visible when the dispensing device is viewed from a front side thereof when the refill is in thermal communication with the heater assembly.

15. The dispenser of claim 14, wherein the refill is visible when the dispensing device is viewed from a left or right side thereof when the refill is in thermal communication with the heater assembly.

16. The dispenser of claim 14, wherein the front portion defines a rounded rectangle periphery.

17. The dispenser of claim 14, wherein the rear portion includes a flanged aperture through which volatile material is configured to release once it has been heated.

18. The dispenser of claim 17, wherein the housing includes one or more rings of dimples at least partially surrounding the flanged aperture.

19. The dispenser of claim 17, wherein the heater assembly is aligned with the flanged aperture.

20. The dispenser of claim 14, wherein the controller is configured to regulate the temperature of the heater assembly based on a size of a room where the dispensing device is located.
